(19) United States
(12) Patent Application Publication
(10) Pub. No.: US 2026/0335246 A1
(43) Pub. Date: Oct. 29, 2026

(54) COMPOSITION OF EXTRACELLULAR VESICLES AND USES THEREOF

(71) Applicants: CONSEJO NACIONAL DE INVESTIGACIONES CIENTÍFICAS Y TÉCNICAS (CONICET), Ciudad Autónoma de Buenos Aires (AR); UNIVERSIDAD NACIONAL DE ROSARIO (UNR), Santa Fe (AR)

(72) Inventors: Susana DELGADO OCAÑA, Rosario (AR); Mercyleidi DÍAZ REYES, Rosario (AR); Dario Esteban MAGAQUIAN, Rosario (AR); Hugo GRAMAJO, Rosario (AR); Claudia BANCHIO, Rosario (AR)

(21) Appl. No.: 19/123,320

(22) PCT Filed: Oct. 26, 2023
(86) PCT No.: PCT/IB2023/060797

§ 371 Date: Apr. 22, 2025

Related U.S. Application Data
(60) Provisional application No. 63/381,013, filed on Oct. 26, 2022.

(51) Int. Cl. A61K 9/50; A61K 35/30; A61P 25/16; C12N 5/0797

(52) U.S. Cl. CPC A61K 9/5068; A61K 35/30; A61P 25/16; C12N 5/0623

(57) ABSTRACT

A composition of extracellular vesicles and uses thereof for the treatment of neurological diseases comprising extracellular vesicles derived from stem cells selected from the group consisting of animal stem cells and cell lines; the extracellular vesicles and a method to obtain the extracellular vesicles. A pharmaceutical formulation wherein the pharmaceutical formulation comprises the composition and a method for treating neurological diseases comprising administering the composition.

### FIELD OF THE INVENTION

The present invention relates to a composition that comprises stem cell-derived extracellular vesicle (EV), a more preferred neural stem cell-derived extracellular vesicle (NSC-EV). The present invention further relates to its uses to promote the proliferation of neural stem cells and its differentiation into neurons under physiological conditions, and restores neuronal plasticity under oxidative stress and inflammatory stress conditions. Also, the present invention further relates to a method to obtain neural stem cell-derived extracellular vesicles. In particular, said composition of neural stem cell-derived EV is to be used in a pharmaceutical composition to treat neurodegenerative diseases.

The present invention relates generally to the field of medicine and neurology. In particular, the invention is directed to treatment of neurodegenerative disorders that lose neurons or neurons parameter associated with their function.

### BACKGROUND

Neural stem cells (NSC) have the potential of self-renewal or differentiation in all the main cell types of the nervous tissue including neurons, astrocytes, and oligodendrocytes (Han et al., 2016). This potential can be strongly influenced by extrinsic signals, including cytokines, growth factors, and neurotrophins (Andreotti et al., 2019).

Neurological disorders and neurodegenerative diseases are caused by, or accompanied with oxidative stress and/or inflammation that causes a major and irreversible loss of neurons (Fischer and Maier, 2015). In this context, mounting evidence supports that stem cell-based therapies could be promising on the basis of their potential for cell differentiation and replacement (Trounson and McDonald, 2015; Han et al., 2016). However, preclinical studies have suggested that very few injected cells (<1%) could survive or differentiate over 4 weeks after transplantation because of the hostile, injured microenvironment (Eggenhofer et al., 2012). In recent years, emerging evidence suggests that EV derived from NSCs is a promising alternative that might ensure more efficient outcomes than stem cell-based therapies (Cossetti et al., 2012, Ma et al., 2019; Zhang et al., 2020) (Mathieu et al., 2019). EVs carry various cell-derived bioactive molecules, including proteins, nucleic acids, lipids, and metabolites (Johnstone, 2006), and deliver functional molecules to recipient cells resulting in the alteration of their physiological and pathological functions (Yokoi and Ochiya, 2021). Thus, various in vitro and in vivo studies have demonstrated that the EVs derived from stem cells display the main features of parent stem cells holding a therapeutic potential (Lai et al., 2010; Xin et al., 2012).

### BRIEF DESCRIPTION OF INVENTION

A first aspect of the present invention provides a composition for the treatment of neurological diseases comprising extracellular vesicles derived from stem cells selected from the group consisting of animal stem cells and cell lines. In a preferred embodiment, said animal stem cell is selected form the group consisting of neural stem cell, mesenchymal stem cell, embryonic stem cell, and umbilical stem cell. In a more preferred embodiment is a neural stem cell. In a more preferred embodiment, said neural stem cell is a mouse neural stem cell. In a more preferred embodiment, the composition of the present invention said neural stem cell is selected from stem cells from brain regions consisting of the lateral portion of the dorsal telencephalon (cortex) of fetal brain tissue, the subventricular zone (SVZ), and the hippocampus. In another embodiment of the present invention, said cell line is selected from the group consisting of neural stem cell line.

In another aspect, the composition of the present invention comprises extracellular vesicles derived from neural stem cells or from human stem cell lines. In another embodiment of the present invention, said extracellular vesicles derived from neural stem cells have a size between 50 nm and 400 nm, more preferred their size is between 100 nm and 200 nm.

In another aspect, the composition the present invention comprises said extracellular vesicles that contain proteins selected from the group consisting of TSG-101, HSP70, ALIX, HSP90, CD81, CD9. In a preferred embodiment, said extracellular vesicle is derived from neural stem cell. In a preferred embodiment of the present invention, said extracellular vesicles further comprise at least one protein selected from the group consisting of Multivesicular body subunit 12B, Vacuolar protein sorting-associated protein 37B, Charged multivesicular body protein 4b. In a more preferred embodiment, said extracellular vesicles comprise proteins selected from the group consisting of TSG-101, HSP70, ALIX, HSP90, CD81, CD9, Multivesicular body subunit 12B, Vacuolar protein sorting-associated protein 37B, Charged multivesicular body protein 4b. In a more preferred embodiment, the composition of the present invention wherein the extracellular vesicles that comprise the proteins selected form the group consisting of TSG-101, HSP70, ALIX, HSP90, CD81, CD9 and further comprises at least one protein selected from the group consisting of Neural cell adhesion molecule 1, Neuronal cell adhesion molecule, Neuronal membrane glycoprotein M6-b, Dihydropyrimidinase-related protein 3, Laminin subunit beta-1, Dihydropyrimidinase-related protein 2, Syntenin-1, Fibroblast growth factor, 2′,3′-cyclic-nucleotide 3′-phosphodiesterase, AP-2 complex subunit alpha-1, MARCKS-related protein, Neuronal membrane glycoprotein M6-a, Neurogenic differentiation factor 6, 14-3-3 protein épsilon, Pleiotrophin, 14-3-3 protein zeta/delta, Epidermal growth factor receptor, Glia-derived nexin, Delphilin, Brevican core protein, Chondroitin sulfate proteoglycan 5, Phosphatidylinositol binding clathrin assembly protein, Tenascin, Tubulin beta-2B chain, Neurocan core protein. In a more preferred embodiment, the composition of the present invention comprises extracellular vesicles that contains proteins selected from the group consisting of TSG-101, HSP70, ALIX, HSP90, CD81, CD9, and further comprise the proteins: Neural cell adhesion molecule 1, Neuronal cell adhesion molecule, Neuronal membrane glycoprotein M6-b, Dihydropyrimidinase-related protein 3, Laminin subunit beta-1, Dihydropyrimidinase-related protein 2, Syntenin-1, Fibroblast growth factor, 2′,3′-cyclic-nucleotide 3′-phosphodiesterase, AP-2 complex subunit alpha-1, MARCKS-related protein, Neuronal membrane glycoprotein M6-a, Neurogenic differentiation factor 6, 14-3-3 protein épsilon, Pleiotrophin, 14-3-3 protein zeta/delta, Epidermal growth factor receptor, Glia-derived nexin, Delphilin, Brevican core protein, Chondroitin sulfate proteoglycan 5, Phosphatidylinositol binding clathrin assembly protein, Tenascin, Tubulin beta-2B chain, Neurocan core. In a more preferred embodiment, the composition of the present invention wherein said extracellular vesicles comprise proteins selected from the group consisting of TSG-101, HSP70, ALIX, HSP90, CD81, CD9 and further comprise at least one protein selected from the group consisting of Vascular cell adhesion molecule 1, Glutathione S-transferase kappa 1, Annexin A1, Thioredoxin, Catalase, Reticulon-3, Peroxiredoxin-2; more preferred comprise the proteins: Vascular cell adhesion molecule 1, Glutathione S-transferase kappa 1, Annexin A1, Thioredoxin, Catalase, Reticulon-3, Peroxiredoxin-2.

In another aspect, the composition of the present invention wherein said extracellular vesicles comprise the proteins selected form the group consisting of TSG-101, HSP70, ALIX, HSP90, CD81, CD9, Multivesicular body subunit 12B, Vacuolar protein sorting-associated protein 37B, Charged multivesicular body protein 4b, Neural cell adhesion molecule 1, Neuronal cell adhesion molecule, Neuronal membrane glycoprotein M6-b, Dihydropyrimidinase-related protein 3, Laminin subunit beta-1, Dihydropyrimidinase-related protein 2, Syntenin-1, Fibroblast growth factor, 2′,3′-cyclic-nucleotide 3′-phosphodiesterase, AP-2 complex subunit alpha-1, MARCKS-related protein, Neuronal membrane glycoprotein M6-a, Neurogenic differentiation factor 6, 14-3-3 protein épsilon, Pleiotrophin, 14-3-3 protein zeta/delta, Epidermal growth factor receptor, Glia-derived nexin, Delphilin, Brevican core protein, Chondroitin sulfate proteoglycan 5, Phosphatidylinositol binding clathrin assembly protein, Tenascin, Tubulin beta-2B chain, Neurocan core protein, Vascular cell adhesion molecule 1, Glutathione S-transferase kappa 1, Annexin A1, Thioredoxin, Catalase, Reticulon-3, Peroxiredoxin-2.

Another aspect of the present invention is to provide an extracellular vesicles comprises the proteins: TSG-101, HSP70, ALIX, HSP90, CD81, CD9, Multivesicular body subunit 12B, Vacuolar protein sorting-associated protein 37B, Charged multivesicular body protein 4b, Neural cell adhesion molecule 1, Neuronal cell adhesion molecule, Neuronal membrane glycoprotein M6-b, Dihydropyrimidinase-related protein 3, Laminin subunit beta-1, Dihydropyrimidinase-related protein 2, Syntenin-1, Fibroblast growth factor, 2′,3′-cyclic-nucleotide 3′-phosphodiesterase, AP-2 complex subunit alpha-1, MARCKS-related protein, Neuronal membrane glycoprotein M6-a, Neurogenic differentiation factor 6, 14-3-3 protein epsilon, Pleiotrophin, 14-3-3 protein zeta/delta, Epidermal growth factor receptor, Glia-derived nexin, Delphilin, Brevican core protein, Chondroitin sulfate proteoglycan 5, Phosphatidylinositol binding clathrin assembly protein, Tenascin, Tubulin beta-2B chain, Neurocan core protein, Vascular cell adhesion molecule 1, Glutathione S-transferase kappa 1, Annexin A1, Thioredoxin, Catalase, Reticulon-3, Peroxiredoxin-2. In a preferred embodiment of the present invention, said extracellular vesicle comprise a size between 50 nm and 400 nm, more preferred its size is between 100 nm and 200 nm.

In another aspect, the composition of the present invention wherein said composition promotes or induces the proliferation of neural stem cells, the neuronal differentiation and the dendritic development. In a more preferred embodiment, the composition of the present invention promotes the proliferation of neural stem cells, neuronal differentiation, and/or neurite development and/or dendritic development and comprises the extracellular vesicles disclosed above.

Another aspect of the present invention is to provide a pharmaceutical formulation that promotes the proliferation of neural stem cells, neuronal differentiation, and/or neurite development and/or dendritic development wherein said pharmaceutical composition comprises the composition of the present invention.

Another aspect of the present invention is to provide a method for treating neurological diseases wherein said method comprises administering the composition of the present invention comprising extracellular vesicles derived from stem cells selected from the group consisting of animal stem cells and cell lines, wherein said diseases are selected from the group consisting of Amyotrophic Lateral Sclerosis, Parkinson's, Alzheimer's, dementia, Huntington disease, Lafora disease, Schizophrenia, Autism, stroke, and from the peripheral nervous system: diabetes, post-chemotherapy paresthesia and pathologies associated with motor, sensory and autonomic nerves. In a more preferred embodiment of the present invention, said extracellular vesicles are derived from neural stem cells. In a more preferred embodiment, said neurological diseases are selected from the group consisting of Parkinson's and strokes.

Another aspect of the present invention is to provide a medicament for treating neurological diseases that comprises the composition of the present invention. In a more preferred embodiment, said medicament is for the treatment of neurodegenerative diseases.

Another aspect of the present invention is to provide a method to obtain extracellular vesicles derived from animal stem cell or stem cell line that comprises culturing the said animal stem cells or cell lines, in a proliferating medium and collecting the fractions containing said extracellular vesicles. In a preferred embodiment, said stem cell is selected from the group consisting of neural stem cells, mesenchymal stem cells, embryonic stem cells, and umbilical stem cells. In a more preferred embodiment, said stem cell is a neural stem cell from brain tissue selected from the group consisting of a lateral portion of the dorsal telencephalon (cortex) of fetal brain tissue, subventricular zone (SVZ), and the hippocampus tissue. In another preferred embodiment of the present invention, said method comprises the steps of: a) isolating a lateral portion of the brain tissue, b) chemically and mechanically disrupting brain tissue into single cells, c) centrifuging, removing the supernatant and resuspending the pellet in serum-free medium, d) culturing cells in a proliferating medium in a culture flask, e) after 7 days, replating cells to obtain a second passage, f) collecting supernatant and centrifuging and filtering to remove cell debris, g) concentrating, separating, and purifying by size exclusion chromatography and h) collecting and pooling fractions and characterizing said neural stem cell-derived extracellular vesicles. In another preferred embodiment, said method wherein said neural stem cell is mouse neural stem cell. In another preferred embodiment of the present invention, said method comprises culturing a cell line in a proliferating medium, collecting supernatant, and collecting fractions containing the extracellular vesicles. In a more preferred embodiment, said cell line is selected from the group consisting of ReNcell; Human Neural stem cell line.

### DESCRIPTION OF THE FIGURES

FIG. 1: NSCs-EV purification and characterization. (A)—Protocol to purify NSC-EVs by sequential centrifugation and size exclusion chromatography. (B)—Size distribution of NSC-EVs measured by DLS. (C)—Electron micrograph of purified NSC-EVs. (D)—Western Blot analysis of purified NSC-EVs. The EVs markers TSG101, HSP70 and ALIX were detected.

FIG. 2: NSCs and neuroblastoma cell incorporate NSC-Derived EV. Dil-NSC-EVs were added to NSC (A) and to human neuroblastoma cells (B), and the uptake of NSC-EVs was immediately monitored by in vivo confocal microscopy (Zeiss LSM 880) and qualitative analyses were performed with Image J”

FIG. 3: Effect of NSC-EV on NSC proliferation. (A) Upper Panel: Quantification of the diameter of the neurospheres under the indicated conditions. The data corresponds to the average+/−SEM of at least three independent experiments. For statistical analysis, a paired Student's T-test was used. * p≤0.05, ** p≤0.01, *** p≤0.001. (B) Image Panel: Representative images of the neurospheres under the indicated conditions obtained through bright-field microscopy (20×). Scale bar: 50 μm.

FIG. 4: NSC-EVs induce NSCs to differentiate into neurons without affecting astroglial differentiation. Representative images (40×) are shown, and the quantification of the (A) percentage of Neurons using the β III Tubulin marker, Astrocytes using the GFAP marker (B), and Oligodendrocytes using the Olig2 marker (C). The graphs correspond to the average+/−SEM from at least three independent experiments. For statistical analysis, the paired parametric Student's test was used. **** p≤0.0001. Scale bar: 50 μm.

FIG. 5: NSC-EVs induce neuronal progenitor proliferation and neuronal differentiation. (A) Illustrative pictures. (B)-Quantification of the % of cells labelled with (C) BrdU and BIII tubulin (neuronal progenitors). For statistical analysis, the paired parametric Student's test was used. p≤0.05, ** p≤0.01. Scale bar: 50 μm.

FIG. 6: Effect of NSC-EV on morphological parameters associated with neuronal function. (A) Quantification of the total length of the main neurite and (B) the total neurite length per cell. The graph corresponds to the average+/−SEM from at least three independent experiments. Statistical analysis was performed using the paired parametric Student's test. * p≤0.05.

FIG. 7: NSC-EVs protects cells from death. (A) Percentage of viable, apoptotic, and necrotic cells obtained by a double staining assay in clone cultures incubated for 48 hours under the indicated conditions. (B) Results represent the mean+/−SD of 3 independent experiments from 10 images each. Statistical analysis was performed using the paired parametric Student's test. * p≤0.05.). Scale bar: 100 μm

FIG. 8: Effect of NSC-EVs on dendritic spine density. (A) Quantification and (B) representative images (63×) of the number of dendritic spines in 20 μm of neurite length. For the immunocytochemistry, the neuronal marker β-III Tubulin was used. The graph shows the average+/−SEM from at least three independent experiments. Statistical analysis was performed using the paired parametric Student's test. ** p≤0.01. Scale bar: 10 μm.

FIG. 9: Effect of NSC-EVs on parameters associated with synapses. (A) Quantification of the number of vesicles containing Synaptophysin (SYP) in 10 μm of neurite length. The graph represents the average±SEM from three independent experiments. (B) Representative images (63×) of the immunocytochemistry using anti-SYP (red) and anti β-III Tubulin (green) antibodies. Scale bar: 1 μm. (C) Representative images and quantification of the Western Blot showing the expression levels of SYP and PSD-95 (postsynaptic protein-95) in NSCs treated or not with the NSC-EVs of the present invention. β-Actin was used as a loading control. The graphs show the densitometry analysis of the bands. Statistical analysis was performed using the paired parametric Student's test. ** p≤0.01 *** p≤0.001.

FIG. 10: Effect of the addition of N2A-EVs and HEK-EVs on the differentiation of neural stem cells. (A) Quantification of the percentage of neurons (Rβ-III Tubulin positive cells) in each condition. The graph corresponds to the average of three independent experiments. For statistical analysis, the ANOVA test was used, followed by the Dunnet test for multiple comparisons. (B) Percentage of normal and aberrant neurons after treatment with N2A-EV. (C) Representative images of the neuronal morphology affected by the treatment with N2A-EV. The graph corresponds to the average+/−SEM of at least three independent experiments. For statistical analysis, the paired parametric Student's test was used. *p<0.05, ***p≤0.0001. Scale bar: 50 μm.

FIG. 11: Effect of H₂O₂ on NSCs viability. Percentage of cell viability under oxidative stress conditions. Cells were incubated for 30 minutes with the indicated concentrations of H₂O₂. Subsequently, the medium was renewed, and the cells were incubated for a 24-hour period. The graph corresponds to the average+/−SEM of at least three independent experiments. Statistical analysis was performed using the ANOVA test, followed by a Tukey test for multiple comparisons. ***p<0.001.

FIG. 12: Effect of NSC-EV treatment on ROS generation induced by H2O2. (A) Representative images (20×) and (B) quantification of the fluorescence of NSCs incubated with DCFH DA under the indicated conditions. (A) Right panel: fluorescence emission (λe: 480) and Left panel: bright-field image as a control. The graph corresponds to the measurement of the relative fluorescence intensity compared to the control. It represents the average+/−SEM of three independent experiments. Statistical analysis was conducted using the paired parametric Student's test. **p<0.01, **** p≤0.0001. Scale bar: 50 μm.

FIG. 13: Effect of NSC EV on NSC proliferation under oxidative stress conditions. (A) Representative images of the neurospheres obtained through bright-field microscopy. (B) Quantification of the diameter of the neurospheres under the indicated conditions. The graph corresponds to the average+/−SEM of at least three independent experiments. For statistical analysis, the ANOVA test was used, followed by the Tukey test for multiple comparisons. *p<0.05, ***p<0.001. Scale bar: 50 μm.

FIG. 14: NSC-EV protects NSCs from oxidative stress. Quantification of the total number of nuclei in 20 photographs of fields chosen at random. The graph corresponds to the average +/−SEM of at least three independent experiments. For statistical analysis, the ANOVA test was used, followed by the Tukey test for multiple comparisons. *p<0.05, ***p<0.001.

FIG. 15: Effect of NSC-EV on neuronal differentiation under oxidative stress conditions. (A) Representative images (40×) and (B) quantification of the percentage of β III Tubulin positive cells (neurons) under the indicated conditions. The graph represents the average+/−SEM of at least three independent experiments. For statistical analysis, the ANOVA test was used, followed by the Tukey test for multiple comparisons. ***p<0.001. Scale bar: 50 μm.

FIG. 16: Effect of NSC-EV on morphological parameters affected by oxidative stress. (A) Total neurite length and (B) total neurites per cell under indicated conditions are shown. The graph represents the average+/−SEM from at least three independent experiments. The statistical analysis was performed using the paired Student's t-test. *p≤0.05.

FIG. 17: Effect of NSC-EV on NSC proliferation under inflammatory stress conditions. (A) Representative images (20×) of neurospheres, obtained through bright-field microscopy. (B) Quantification of neurosphere diameter under indicated conditions. The graph represents the average+/−SEM from at least three independent experiments. ANOVA test was used for statistical analysis. Scale bar: 50 μm.

FIG. 18: Effect of NSC-EV on neuronal differentiation under inflammatory stress conditions. (A) Representative images (40×) and (B) quantification of the percentage of β-III Tubulin positive cells (neurons) under indicated conditions. The graph represents the average+/−SEM from at least three independent experiments. The statistical analysis was performed using the ANOVA test, followed by the Tukey test for multiple comparisons. ****p<0.0001. Scale bar: 50 μm.

FIG. 19: Effect of NSC-EV on neuronal morphology affected by inflammatory damage. (A) Representative images (100×) and (B) quantification of the percentage of normal and aberrant neurons under indicated conditions. Immunocytochemistry was performed, and neurons were marked with the β-III Tubulin marker. The percentage of normal and aberrant neurons was quantified in relation to the total neurons. The graph represents the average +/−SEM from three independent experiments. ANOVA followed by Tukey's test was used for statistical analysis. *p<0.05. Scale bar: 50 μm.

FIG. 20: Effect of NSC-EV of the present invention on dendritic spine density under stress conditions. (A) Representative images (63×) showing the number of dendritic spines in 20 μm of neurite under indicated conditions (H2O2 50 μM or 20% of activated macrophage medium: AMM) in the presence or absence of NSC EV for 72 hours (B). The graph represents the average+/−SEM from at least three independent experiments. ANOVA followed by Tukey's test was used for statistical analysis. *p<0.05, **p<0.01. Scale bar: 10 μm.

FIG. 21: Effect of NSC-EV on SYP vesicles density under stress conditions. (A) Representative images (63×) and (B) quantification of the number of vesicles containing SYP in 10 μm of neurite length under indicated conditions. The graph represents the average+/−SEM from three independent experiments. ANOVA followed by Tukey's test was used for statistical analysis. *p<0.05, ***p<0.001. Scale bar: 2 μm.

FIG. 22: Distinct proteins in NSC-EV: Representation by Venn Diagram. Comparison of proteins identified in NSC-EVs of the present invention in relation to those in the Exocarta database and those identified in exosomes derived from mesenchymal stem cells (MSC EV) is shown.

FIG. 23: Effect of α-Synuclein overexpression on cell survival and ROS accumulation. (A) MTT assay of clone cultures incubated for 72 hours under differentiation conditions. (B) Percentage of viable, apoptotic, and necrotic cells obtained by a double staining assay in clone cultures incubated for 72 hours under differentiation conditions. Results represent the mean+/−SD of 3 independent experiments from 10 images each (One-Way ANOVA; Dunnett; p<0.01). (C) Effect of α-Synuclein protein overexpression on ROS generation through DCFH-DA fluorescence quantification. Results represent the mean+/−SD of 3 experiments (One-Way ANOVA; Dunnett; p*<0.05, p**<0.01, p***<0.001, p****<0.0001) pcDNA: SH-SY5Y pcDNA3 clone 1; WT: SH-SY5Y pcDNA3-WT-α-Synuclein clone 1; A53T: SH-SY5Y pcDNA3-A53T-α-Synuclein clone 1.

FIG. 24: Effect of NSC-EV on the survival and ROS accumulation of cells overexpressing α-Synuclein. (A) MTT assay of clone cultures incubated for 72 hours under differentiation conditions in the presence or absence of NSC-EV from the present invention. (B) Effect of NSC-EV from the present invention on ROS generation in clones overexpressing the α-Synuclein protein, through the quantification of DCFH-DA fluorescence. Results represent the mean+/−SD of 3 experiments (One-Way ANOVA; Dunnett; * p<0.05, ** p<0.01). pcDNA: SH-SY5Y pcDNA3 clone 1; WT: SH-SY5Y pcDNA3-WT-α-Synuclein clone 1; A53T: SH-SY5Y pcDNA3-A53T-α-Synuclein clone 1.

FIG. 25: Effect of 6-hidroxidopamine (6-OHD) on cell viability in cultures incubated under differentiation conditions. Percentage of cell viability obtained using the MTT assay in SH-SY5Y cell cultures incubated under differentiation conditions and treated with the indicated concentration of 6-OHD. Results represent the mean of 3 independent experiments+/−SD, each performed in triplicate (One-Way ANOVA; Dunnett; different letters represent significant differences). 6-OHD: 6-hydroxydopamine. 10 μM doses of 6-OHD was selected to establish the PD model.

FIG. 26: Effect of 6-OHD on cell death and ROS accumulation. (A) Percentage of viable, apoptotic, and necrotic cells obtained by a double staining assay of SH-SY5Y cell cultures incubated under differentiation conditions and treated with 10 μM 6-OHD. (B) Effect of 60HD on ROS generation, through the quantification of DCFH-DA fluorescence. Results represent the mean+/−SD of 3 independent experiments from 10 images each (t-test, *** p<0.001; **** p<0.0001). 60HD: 6-hydroxydopamine.

FIG. 27: Effect of pre-incubation of cells with NSC-EV of the present invention against the damage caused by 6-OHD in cultures incubated under differentiation conditions. (A) Timeline representation of the experiment protocol. (B) MTT assay in SH-SY5Y cell cultures grown under differentiation conditions, pre-incubated with NSC-EV from the present invention, and treated with 10 μM 60HD. (C) ROS generation in SH-SY5Y cells grown under differentiation conditions, pre-incubated with NSC-EV from the present invention, and treated with 10 μM 6-OHD. Results represent the mean+/−SD of 3 independent experiments, each performed in triplicate (One-Way ANOVA; Tukey; * p<0.05; ** p<0.01; *** p<0.001). 6-OHD: 6-hydroxydopamine.

FIG. 28: Restorative effect of NSC-EV on cells treated with 6-OHD. (A) Timeline representation of the experiment protocol. (B) MTT assay in SH-SY5Y cell cultures grown under differentiation conditions, treated with 10 μM 6-OHD, and incubated with NSC-EV. (C) ROS generation in SH-SY5Y cells grown under differentiation conditions, treated with 10 μM 6-OHD, and incubated with NSC-EV. Results represent the mean+/−SD of 3 independent experiments, each performed in triplicate (One-Way ANOVA; Tukey; * p<0.05; ** p<0.01; **** p<0.0001). 60HD: 6-hydroxydopamine.

FIG. 29: Effect of various concentrations of NSC-EV on cell survival. Percentage of cell viability obtained using the MTT assay in SH-SY5Y cell cultures incubated under differentiation conditions* p<0.05,** p<0.01,** p<0.001).

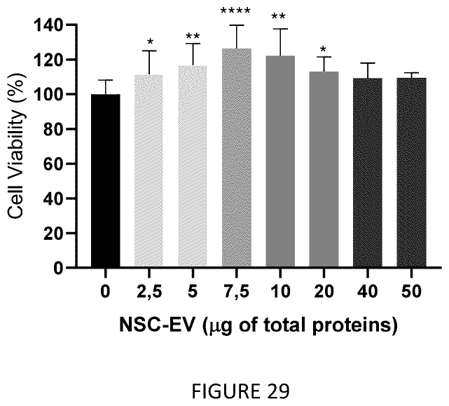

### DETAILED DESCRIPTION OF THE INVENTION

As used herein, the term extracellular vesicle (also known as EV, EVs, NSC-EV, NSC-EVs), is defined as vesicle derived from a stem cell or a cell line, capable of promoting or inducing the restoration of neuronal plasticity in neurological diseases associated with the central nervous system or peripheral nervous system. Particularly, NSC-EV or NSC-EVs are “extracellular vesicles derived from neural stem cell” or “neural stem cell-derived extracellular vesicles”.

As used herein, stem cell is defined as undifferentiated cells that have the ability to self-renew and give rise to different cell types that make up an organism. These stem cells can be totipotent, pluripotent, or multipotent type. Also, stem cells can be classified according to their origin: embryonic if they come from an embryonic organism or adult stem cells if they come from an adult organism tissue. Said stem cell can be neural stem cells, mesenchymal stem cells, embryonic stem cells, umbilical stem cells.

Neural stem cells (NSCs) are defined as multipotent stem cells that have the ability to generate the three main types of cells in the central nervous system (CNS): neurons, astrocytes, and oligodendrocytes.

As used herein, a cell line is defined as an immortalized neural stem cell line with the ability to easily differentiate into neurons and/or glial cells, such as ReNcell (R) Human Neural Stem Cell (iPSC-derived-iXCells), Human Neural Stem Cell (Cell Applications), Neural Progenitor Cells (ATCC), Human Neural Stem Cell Line (ALSTEM), Human Neural Stem Cell Line (Creative Biolabs), iPSC Differentiated Neural Stem Cells (Applied Stem Cell).

As used herein, the term neuronal plasticity is defined as the ability of the nervous system to change its structure and function, understood as the adaptive potential of the nervous system that allows the brain to recover from disorders or injuries, and can reduce the effects of structural alterations produced by pathologies associated with neurological impairment and/or neurodegenerative processes. Recovery of neuronal plasticity is understood as a therapeutic process that promotes neural stem cell proliferation, neuronal differentiation, dendritic development, synaptic development or a combination of them.

The inventors have demonstrated that EVs derived from NSCs (NSC-EVs) promote NSCs proliferation under control conditions and also rescue this capacity under oxidative stress. They also show that EVs can influence the process of NSCs differentiation under normal and stress conditions common to many neurodegenerative diseases: NSC-EVs promote neuronal differentiation without affecting astroglial differentiation. More importantly, NSC-EVs treatment restores the aberrant phenotype induced by inflammation by increasing morphological and functional parameters involved in neuronal function and synapses. In conclusion, NSC-EVs could be used for the treatment of diseases that are accompanied by the loss of neurons or by defects in neuronal parameters associated with their function.

The extracellular vesicles, which are part of the composition of this invention, derived from neural stem cells, have a size distribution between 50 nm and 400 nm, with the predominant size being those vesicles between 100 nm and 200 nm (SEE EXAMPLE 1).

The proteomics of the extracellular vesicles that are part of the composition of the present invention includes a set of proteins that are fundamental components for the inducing and/or promoting effect of neuronal plasticity (see example 14).

Some of the proteins identified in the proteomics, such as those selected from the group comprising TSG-101, HSP70, ALIX (also known as Programmed cell death 6-interacting protein), HSP90, CD81, CD9; are recognized in the literature as marker proteins of extracellular vesicles. This set of proteins is involved in the biogenesis and secretion of extracellular vesicles and, in addition, proteins selected from the group comprising Multivesicular body subunit 12B, Vacuolar protein sorting-associated protein 37B, charged multivesicular body protein 4b were also detected in these particular extracellular vesicles, which would also be involved in the same function.

Furthermore, other specific proteins were identified in the extracellular vesicles, that are part of the composition of the present invention, and which are not found in other types of extracellular vesicles or other extracellular structures, that do not achieve the same effects of repairing neurological pathologies, particularly neurodegenerative ones. Said proteins are selected from the group comprising Neural cell adhesion molecule 1, Neuronal cell adhesion molecule, Neuronal membrane glycoprotein M6-b, Dihydropyrimidinase-related protein 3, Laminin subunit beta-1, Dihydropyrimidinase-related protein 2, Syntenin-1, Fibroblast growth factor, 2′,3′-cyclic-nucleotide 3′-phosphodiesterase, AP-2 complex subunit alpha-1, MARCKS-related protein, Neuronal membrane glycoprotein M6-a, Neurogenic differentiation factor 6, 14-3-3 protein epsilon, Pleiotrophin, 14-3-3 protein zeta/delta, Epidermal growth factor receptor, Glia-derived nexin, Delphilin, Brevican core protein, Chondroitin sulfate proteoglycan 5, Phosphatidylinositol binding clathrin assembly protein, Tenascin, Tubulin beta-2B chain, Neurocan core protein, Vascular cell adhesion molecule 1, Glutathione S-transferase kappa 1, Annexin A1, Thioredoxin, Catalase, Reticulon-3, Peroxiredoxin-2.

In a preferred embodiment of the present invention, the extracellular vesicles of the present invention are characterized by containing a set of proteins selected from the group comprising Neural cell adhesion molecule 1, Neuronal cell adhesion molecule, Neuronal membrane glycoprotein M6-b, Dihydropyrimidinase-related protein 3, Laminin subunit beta-1, Dihydropyrimidinase-related protein 2, Syntenin-1, Fibroblast growth factor, 2′,3′-cyclic-nucleotide 3′-phosphodiesterase, AP-2 complex subunit alpha-1, MARCKS-related protein, Neuronal membrane glycoprotein M6-a, Neurogenic differentiation factor 6, 14-3-3 protein epsilon, Pleiotrophin, 14-3-3 protein zeta/delta, Epidermal growth factor receptor, Glia-derived nexin, Delphilin, Brevican core protein, Chondroitin sulfate proteoglycan 5, Phosphatidylinositol binding clathrin assembly protein, Tenascin, Tubulin beta-2B chain, Neurocan core protein. These proteins are involved in the proliferation and differentiation of NSCs as well as the maturation and growth of neurons and synaptic function.

In another preferred embodiment of the present invention, the extracellular vesicles of the present invention are characterized by containing a set of proteins selected from the group comprising Vascular cell adhesion molecule 1, Glutathione S-transferase kappa 1, Annexin A1, Thioredoxin, Catalase, Reticulon-3, Peroxiredoxin-2. These proteins have a protective and repairing effect on neuronal morphology and function even under conditions of oxidative and inflammatory stress.

In another embodiment of the present invention, the composition comprising extracellular vesicles derived from stem cell or cell line, of the present invention, can be used for the treatment of neurological diseases of various origins, such as neurological diseases associated with pathologies at the level of the central nervous system, as well as those neurological diseases associated with pathologies at the peripheral nervous system level.

In another embodiment of the present invention, the composition of the present invention can be used for the treatment of neurological diseases such as Stroke, Amyotrophic Lateral Sclerosis, Parkinson's, Alzheimer's, dementia, Huntington's disease, Lafora disease, Schizophrenia, Autism, among others, associated with the central nervous system and characterized by their progressive neurodegenerative nature.

In another embodiment of the present invention, the composition of the present invention can be used for the treatment of neurological diseases of the peripheral nervous system as well as neurological pathologies associated with diabetes, post-chemotherapy paresthesia, and pathologies associated with motor, sensory, and autonomic nerves.

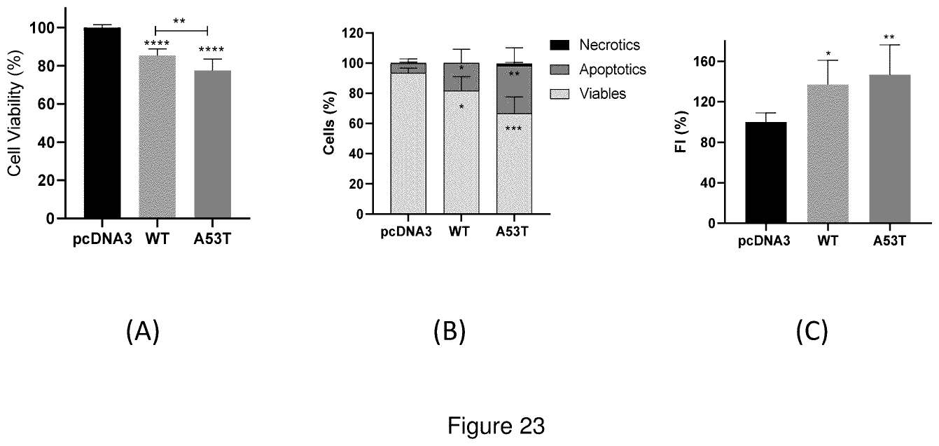

The composition of the present invention comprising the extracellular vesicles wherein said extracellular vesicles comprises the set of proteins identified, in the proteomics according to EXAMPLE 14, has an inducing effect on neuronal differentiation, promotes the proliferation and differentiation of neural stem cells, induces and promotes dendritic development, restoring synaptic function. Additionally, it has a protective and repairing effect on neuronal morphology and function even under conditions of oxidative and inflammatory stress, and also induces the survival in PD model (FIG. 23).

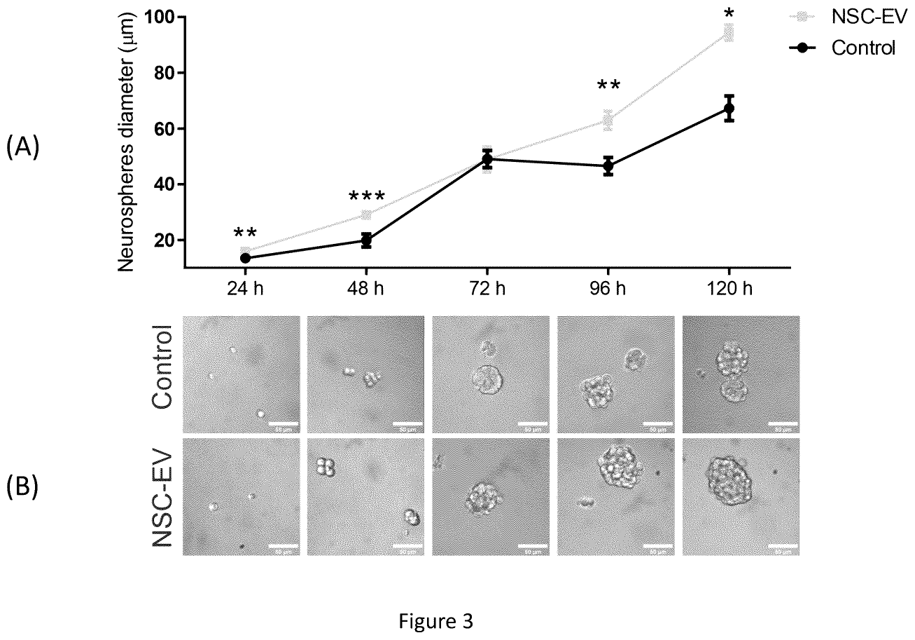

The composition of the present invention can be used to prevent neurodegenerative disease as NSC-EVs induce neuronal differentiation and increase functional neuronal parameters under physiological conditions (FIGS. 3-7).

The composition of the present invention can be used for the recovery of neuronal plasticity in therapeutic processes associated with pathologies of the nervous system.

To evaluate the technical effects of the composition of the present invention, the inventors have carried out a series of tests in order to evaluate the potential of inducing the proliferation of neural cells contributing to the restoration of neural plasticity. It is known that the self-renewal of NSCs and the maintenance of their proliferative potential are fundamental characteristics to maintain the NSC reservoirs and the regenerative capacity of the brain, defined as cognitive reserve. In this context, the composition of the present invention that comprises extracellular vesicles derived from NSCs induce the proliferation of NSCs, measured as a significant increase in the diameter of the neurospheres.

Various analyses carried out by the inventors in the present invention, show that the extracellular vesicles that are part of the composition of the present invention, induce the differentiation of the neuronal lineage, measured as an increase in the percentage of cells positive for the neuronal marker β-III Tubulin, without affecting differentiation towards the astroglial or oligodendroglial lineage. This could be due to three possible pathways: a) that the composition comprising extracellular vesicles of the present invention accelerates the process of neuronal differentiation; b) that it exerts a neuroprotective effect or; c) that they induce an increase in the proliferation of neural precursors.

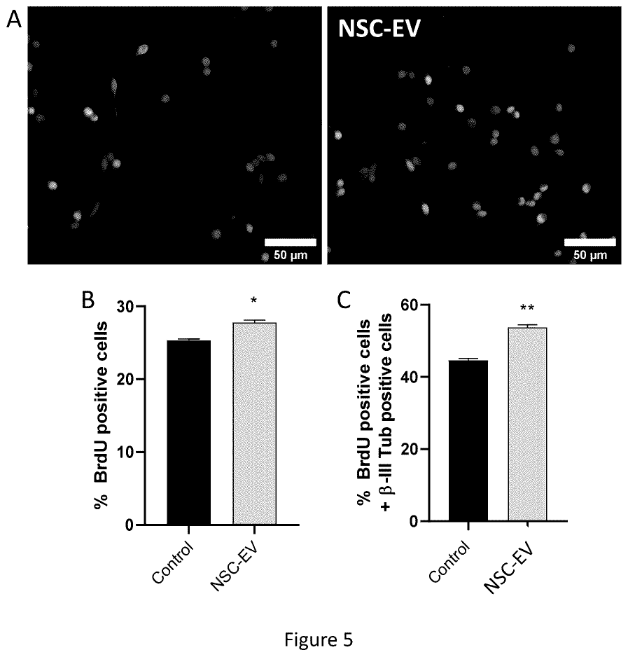

On the other hand, the inventors demonstrated from different tests that the treatment with the composition of the present invention induces neuronal differentiation by inducing specifically the proliferation of neuronal progenitors (FIG. 5).

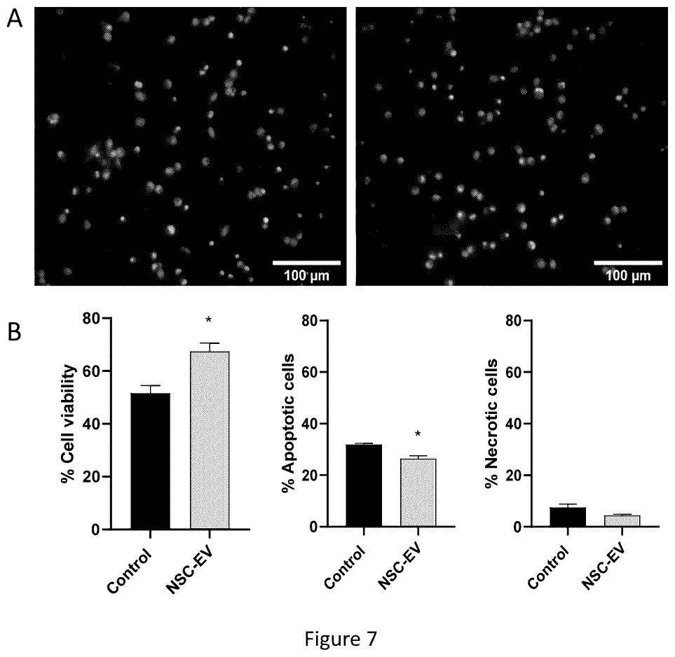

On the other hand, the inventors demonstrated from different tests that the treatment with the composition of the present invention increases the percentage of viable cells and decreases the percentage of apoptotic cells. These results suggest that the composition of the present invention exerts a protective effect, preventing the death of neural progenitors or developing neurons (FIG. 7).

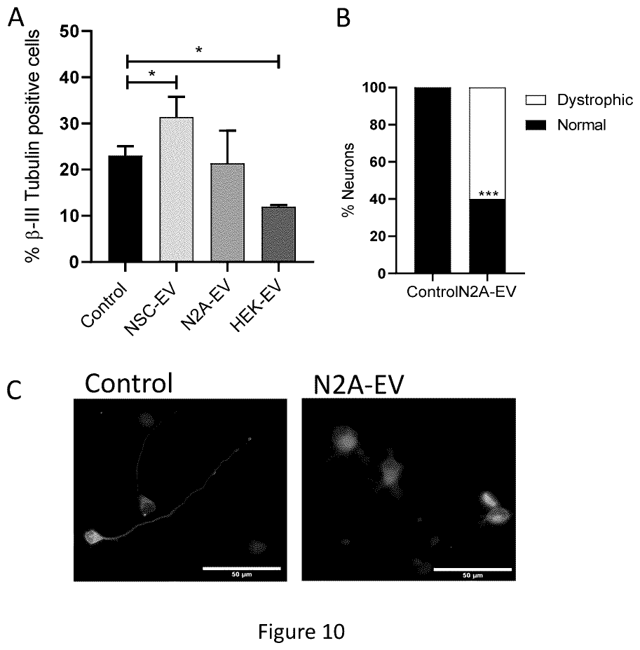

The inventors have also found that the inducing effect on neuronal differentiation, observed with the use of the extracellular of the present invention, was not observed in NSCs treated with extracellular vesicles derived from the HEK 293 and Neuro2A cell lines. This shows that NSC-EVs of the present invention contain specific bioactive components that are not present (or are present in lesser abundance) in extracellular vesicles from other cell types (FIG. 10).

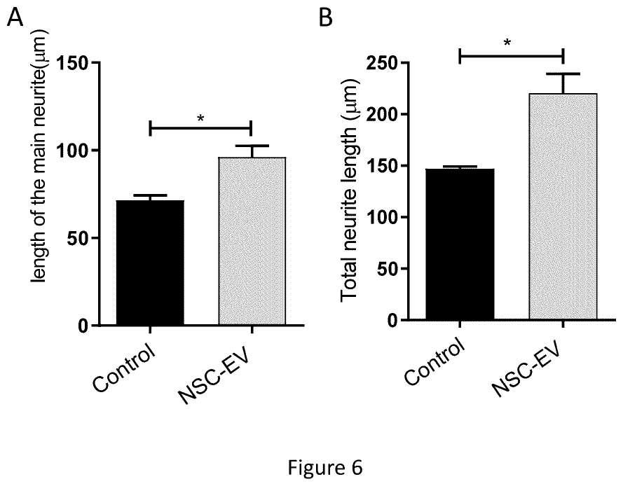

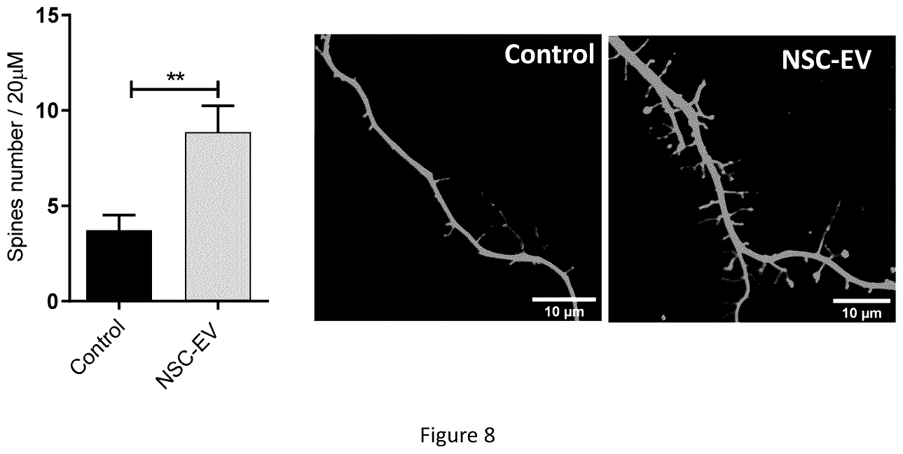

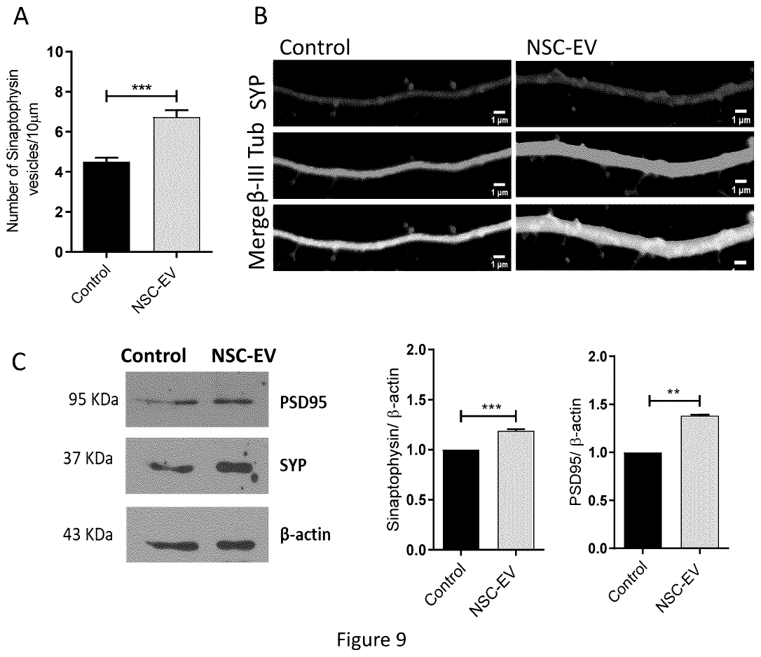

Furthermore, in addition to this effect on neuronal differentiation, the inventors demonstrated that the treatment with the composition of the present invention promotes the development of morphological parameters associated with neuronal functionality, such as total neurite length per cell, main neurite length (FIG. 6), dendritic spine density (FIG. 8), and the number and expression of pre- and post-synaptic proteins SYP and PSD 95 (FIG. 9).

The inventors demonstrated that the treatment with the composition of the present invention, which contains the extracellular vesicles, protects NSCs under oxidative stress conditions, maintaining the proliferation capability of the NSCs. Interestingly, the treatment with the composition of the present invention restores the proliferation and survival of NSCs to levels comparable to the controls. The effects of the composition of the present invention are highly relevant and beneficial as they preserve cognitive reserve even under stress conditions. This might be partly because they reduce the generation of ROS, which affects neuronal maturation and development.

More interestingly, treatment with the composition containing extracellular vesicles of the present invention restores the differentiation capability of NSCs towards functional neurons in environments under oxidative stress conditions, which is essential for repairing damaged tissue.

Surprisingly, the inventors have shown that in a model of oxidative stress conditions, like in Parkinson's, the treatment with the composition of the present invention promotes the morphological recovery of neurons, favoring differentiation towards regular neurons, reducing the aberrant phenotype caused by inflammatory damage.

In addition, the inventors also demonstrated that under inflammatory stress, which has a deleterious effect on the morphological parameters associated with synaptic functionality, the treatment with the composition of the present invention, induces the recovery of these morphological parameters (dendritic spine density, the number of vesicles containing SYP and the expression of synaptic proteins SYP and PSD-95) to values comparable to the control. This result shows the potential restorative effect of the NSC-EV of the present invention under damage conditions like inflammatory damage conditions. The inventors have demonstrated that the treatment with the composition of the present invention reverses the detrimental effect on neuronal morphology and functionality under inflammatory damage conditions.

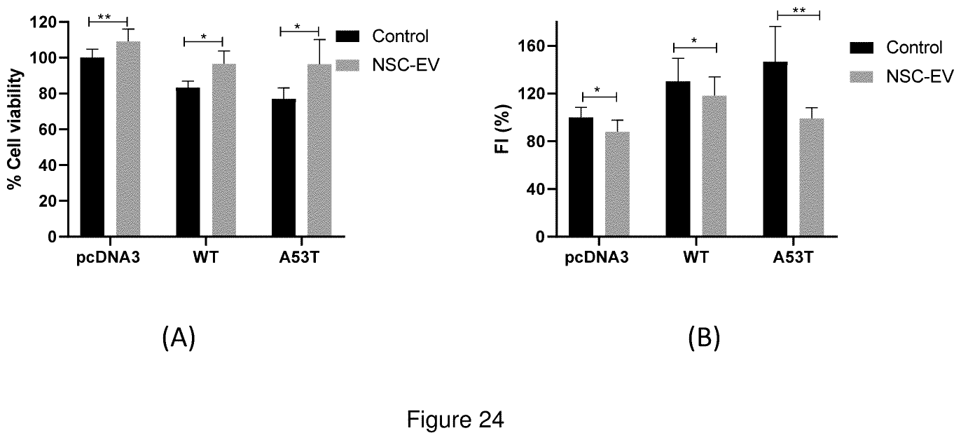

In addition, it was demonstrated in the Parkinson's disease model (both by Synuclein overexpression and by 6-OHDA treatment), that NSC-EVs increase survival, reduce ROS accumulation and prevent or restore the damage (FIGS. 23, 24, 27, 28).

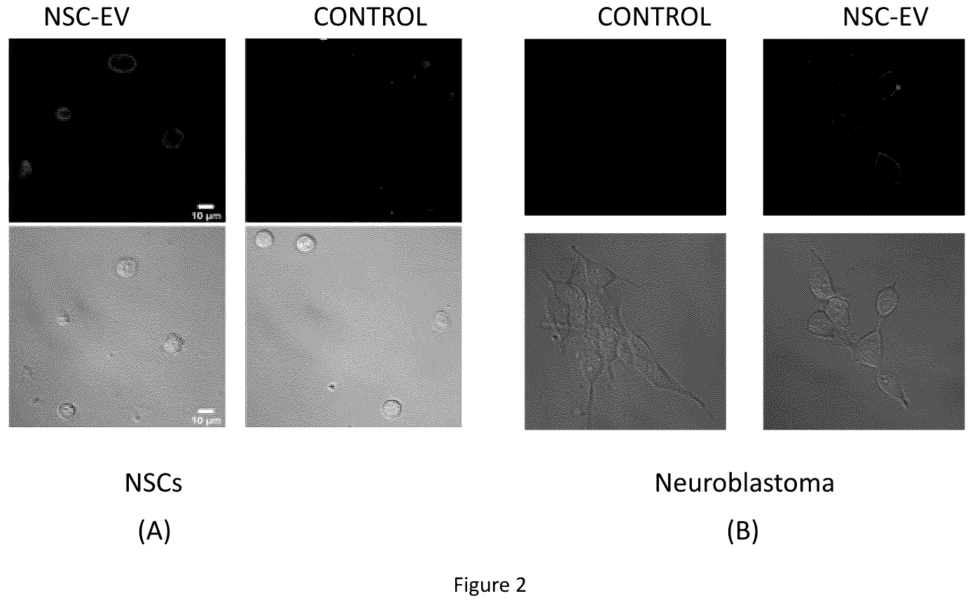

This human neuroblastoma cell line incorporates NSC-EV from mouse NSC indicating an interspecies effect (FIG. 2).

The following examples are provided to further demonstrate and illustrate certain embodiments and aspects of the present invention and should not be construed as limiting its scope.

### EXAMPLES

### Example 1: Isolation and Culture of Fetal Neural Stem Cells

Neural stem cells (NSC) were obtained from mouse fetal brain tissue as previously described (Costa et al., 2008). Briefly, the lateral portion of the dorsal telencephalon (cortex) of embryonic day 13 (E13) C57/BL6 mice were isolated and chemically disrupted with trypsin (5 minutes, 0.05% w/v) and then mechanically disrupted into single cells by repeated pipetting in medium DMEM/F12 (1:1) containing 10% FBS, penicillin G (100 units/ml) and streptomycin (100 μg/ml). After centrifugation (5 minutes, 100 rpm) the pellet was resuspended in serum-free medium DMEM/F12 (1:1) and dissociated cells were cultured at a density of 5×10⁴ cells/ml in proliferation medium containing DMEM/F12 (1:1) supplemented with B27, 10 ng/ml bFGF and 10 ng/ml EGF and maintained at 37 °C in a humidified 5% CO₂ incubator. After 7 days, primary neurospheres were collected by low speed centrifugation and dissociated chemically and mechanically into single cells. Then cells were re-plated to obtain a new passage: all the experiments were performed using cells from the second passage.

Neural stem cell differentiation was performed by plating 2.5×10⁵ or 5×10⁴ single cells on poly-D-lysine (PDL) (10 μg/ml)-coated 24 or 96 well plates respectively in differentiation medium containing DMEM/F12 (1:1) supplemented with B27.

All animal experiments and related protocols were approved by the Bioethics Commission for the Management and Use of Laboratory Animals of National University of Rosario, Argentina (N 6060/89). All procedures were carried out in accordance with the approved guidelines (Guide for the care and use of Laboratory Animals—8° edition—e National Academies Press-Washington DC 2011 and in compliance with the ARRIVE guidelines).

### Example 2: Extracellular Vesicles Isolation, Purification and Characterization

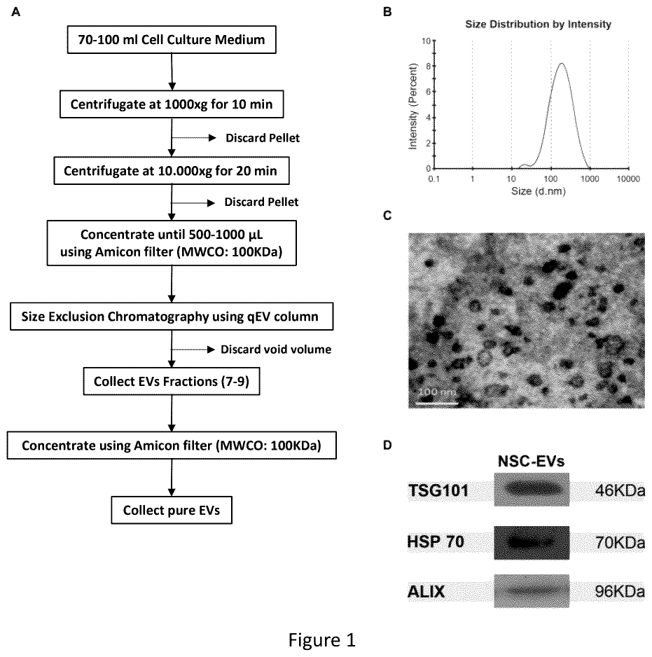

NSC-EVs were purified using size exclusion chromatography (IZION qEV original) according to the manufacturer's instructions (FIG. 1A). Briefly, NSCs supernatants were centrifuged at 500 g for 10 min and then 10,000 g for 10 min to remove cell debris and passed through a 0.22 μm filter to remove larger vesicles and cellular debris. Cleared conditioned media was concentrated to a final volume of 500-1000 μl using AmiconR Ultra-15 100,000 kDa Centrifugal Filters (Millipore Sigma). NSC-EVs separation was performed after column equilibration at room temperature. Fractions of 0.5 ml were collected using PBS as eluent. Four EV-rich fractions (8-11) were pooled and either analysed directly or concentrated using an AmiconR Ultra-4 10 kDa centrifugal filters. Negative staining of NSC-EV suspensions followed by imaging in a transmission electron microscope (TEM) was used to determine vesicle shape and size distribution. 10 ul aliquots of EVs suspensions were dispensed onto sheets of Parafilm in a humidified petri dish and the vesicles were deposited on carbon-coated grid (300-400 mesh) for 3-5 min. Afterward, the sample was negatively stained with 2% uranyl acetate for 3-5 min. The excess stain was removed with filter paper and the grid was briefly washed with Mili-Q water and air-dried for 15 min at RT. Images were taken using a Zeiss EM 109T accoupled to a digital camera Gatan ES 1000W. In addition, dynamic light scattering (DLS) was performed to determine the size of EVs.

### Characterization

Dynamic light scattering (DLS) revealed that 97,2% of the purified NSC-EVs have an average size of 164.2 nm (FIG. 1B), and transmission electron microscopy (TEM) analysis showed their donut-shaped morphology (FIG. 1C). The inventors also looked for the presence of NSC-EVs proteins like Alix, TSG101 and HSP70 (Witwer et al., 2013) by Western Blot (FIG. 1D). Taken together, the foregoing data suggest that the method allows the efficient and straight-forward isolation of NSC-EVs. In addition, morphological and proteomic analyses confirmed that they are suitable for use in the subsequent assays and experiments.

Mouse NSCs and human neuroblastoma take labelled NSC-EVs. For labelling, 25 ul of NSC-EVs in PBS were mixed with Vybrant™ DiI Cell-Labelling Solution (Thermo Fisher) (1:9) and incubated at room temperature for 30 min. Next, Dil-NSC-EVs were added to NSC and the uptake of NSC-EVs was immediately monitored by in vivo confocal microscopy (Zeiss LSM 880), and qualitative analyses were performed with Image J” (NIH). (FIG. 2)

### Example 3: Effect of NSC-EVs on Neural Stem Cell Proliferation

The proliferation and self-renewal of neural stem cells (NSCs) are fundamental characteristics that contribute to the maintenance of stem cell pool, which means preserving cognitive reserves and maintaining the regenerative potential of the brain tissue. Considering that extracellular vesicles act as intercellular messengers, and keeping in mind that the extracellular vesicles of the present invention, obtained according to examples 1 and 2, are taken up by NSCs (FIG. 2), it was examined whether they could act as an autocrine signal regulating stem cell proliferation. NSCs grow forming spheres in suspension called neurospheres, which are formed from a single stem cell that proliferates to form a cell mass. Therefore, their diameter is proportional to cell proliferation and is used to measure this parameter. To evaluate the effect of the extracellular vesicles of the present invention on NSC proliferation, 5,000 cells obtained from the second passage were seeded and grown under proliferative conditions for 120 hours in the presence or absence of the extracellular vesicles that are part of the composition of the present invention (7.5 μg of protein from the extracellular vesicles of the present invention). Every 24 hours, 20 random photos were taken to quantify the diameter of the neurospheres using the free ImageJ software. In this way, a growth curve was obtained in the presence or absence of the extracellular vesicles of the present invention.

As observed in the micrographs and in the resulting graph of the quantification, the addition of extracellular vesicles that are part of the composition of the present invention significantly increases the diameter of the neurospheres, indicating that supplementation of the culture medium with said extracellular vesicles obtained according to example 2, induces NSC cell proliferation (FIG. 3).

### Example 4: Effect of NSC-EVs on Neural Stem Cell Differentiation

The balance between the proliferation and differentiation of NSCs is essential for maintaining the homeostasis of the nervous tissue. To study the role of NSC-EVs in this process, 250,000 NSCs were seeded under differentiation conditions, treated with extracellular vesicles derived from neural stem cells obtained according to example 2 (7.5 μg of EV proteins), and incubated for a period of 72 hours. After this time, an immunocytochemistry assay was conducted using the neuronal marker β III Tubulin. For quantitative analysis, 20 random field photos were taken with the Nikon Eclipse 800 microscope, and the percentage of β III Tubulin positive cells relative to the total number of cells (nuclei stained with Hoechst) was analyzed.

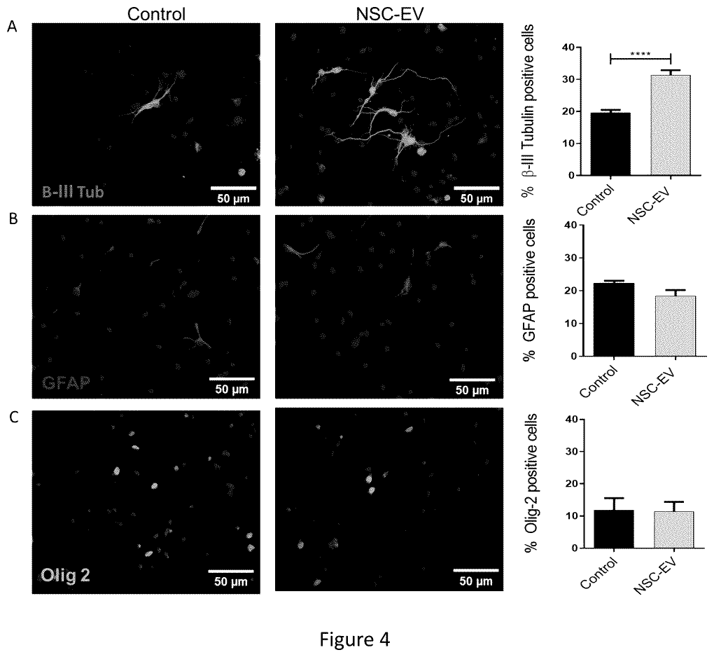

As shown in FIG. 4A, treatment with NSC-EVs, obtained according to example 2, leads to a significant increase in the number of β III Tubulin positive cells compared to the control, indicating that the NSC-EVs of the present invention increases the differentiation of NSCs towards the neuronal lineage.

To evaluate the effect of the extracellular vesicles of the present invention on differentiation towards the astroglial or oligodendroglial lineage, the same experiment was carried out, but the percentage of astrocytes labeled with GFAP or oligodendrocytes labeled with Olig 2 was analyzed. It was observed that treatment with these extracellular vesicles does not affect the percentage of GFAP positive cells or Olig 2 positive cells, indicating that these extracellular vesicles do not influence differentiation towards the astroglial or oligodendroglial lineage respectively (FIG. 4B-C).

### Example 5: Effect of NSC-EVs on Morphological and Functional Parameters Associated with Neuronal Function and Synapses

The morphology and function of the neurons were assessed. 250,000 cells were seeded under differentiation conditions and incubated for 72 hours in the presence or absence of NSC-EV of the present invention obtained from example 2 (7.5 μg of EV proteins). After this period, immunocytochemistry was conducted in the same manner previously described, using the neuronal marker β-III Tubulin. Twenty random field photos were taken, and the total length of the primary neurite and the total neurite length per cell were quantified using the free ImageJ software. The results show that NSC-EV treatment induces a significant increase in both morphological parameters (FIG. 6).

Since neuronal morphology is linked to function, it was decided to analyze whether the NSC-EV of the present invention affects parameters associated with synaptic function. Synapses are specialized intercellular connections through which one neuron connects and communicates with another, enabling nerve stimulus transmission. Axon-dendrite contact occurs through specialized projections of the dendrite called dendritic spines. In addition to spines, various proteins present at the synaptic terminal, such as the presynaptic protein Synaptophysin (SYP), which is a fundamental component of synaptic vesicles, and the postsynaptic density protein PSD-95, are involved in this process. Therefore, evaluating how these factors change is an indirect way of assessing synaptic function.

To analyze these parameters, the same protocol previously described was followed, involving incubating NSCs under differentiation conditions for 72 hours, in the presence or absence of NSC-EV. Subsequently, immunocytochemistry was performed using anti β-III Tubulin and anti-SYP antibodies. Images were taken using a confocal microscope (Zeiss LSM 880) and analyzed with the ImageJ software.

The number of dendritic spines in 20 μm of length and the number of vesicles containing SYP in 10 μm of neurite length were quantified. As seen in FIG. 8, supplementing the culture medium with NSC-EV from the present invention significantly increases the density of dendritic spines.

Further analysis revealed that the number of vesicles containing SYP increases under treatment conditions with NSC-EV of the present invention, obtained from example 2, compared to the control condition (FIG. 9 A-B). This result was corroborated by measuring the expression of SYP and PSD-95 using Western Blot analysis. For this, a lysate of NSCs grown under differentiation conditions for 72 hours in the presence or absence of the NSC-EV of the present invention was made. 15 μg of total proteins for each condition were seeded, and the anti-SYP, anti PSD-95, and loading control anti R-Actin antibodies were used. The results demonstrate that treatment with NSC-EV of the present invention increases the expression of the SYP protein, as well as the postsynaptic PSD-95 protein (FIG. 9C).

### Example 6: Effect of EVs Derived from Other Cell Types on NSCs Differentiation

With the aim of determining if the previously demonstrated neuron differentiation-inducing effect is exclusive to EVs derived from stem cells of the current invention, particularly from the neural stem cells obtained from Example 2, or if it's caused by EVs derived from other cell types. To answer this question, Extracellular Vesicles from the murine neuroblastoma cell line Neuro-2a (N2A) and the HEK-293 cell line (Human embryonic kidney cells) were purified and their effect under differentiation conditions was analyzed in the same manner as previously explained for EVs derived from neural stem cells, but supplementing the culture medium with 7.5 μg of N2A-EV or HEK-EV. The results show that both N2A-EV and HEK-EV do not induce neuronal differentiation (FIG. 10A). Furthermore, the morphological analysis shows that supplementing the culture medium with N2A-EV affects neuronal morphology, inducing an aberrant phenotype where the β-III tubulin positive neurons display irregular somas and short, dystrophic neurites (FIG. 10B-C).

### Example 7: Establishment of the In Vitro Oxidative Stress Model

Given that oxidative stress and inflammation are two common characteristics that have been associated with the progression of various degenerative diseases and disorders of the nervous system, it was evaluated how these conditions affect proliferation and neuronal differentiation. In this scenario and considering the results that show that treatment with NSC-EV of the current invention promotes neuronal proliferation and differentiation, it was evaluated if, under conditions that mimic damage to the nervous system, the NSC-EVs of the current invention are capable of inducing differentiation and neuronal plasticity.

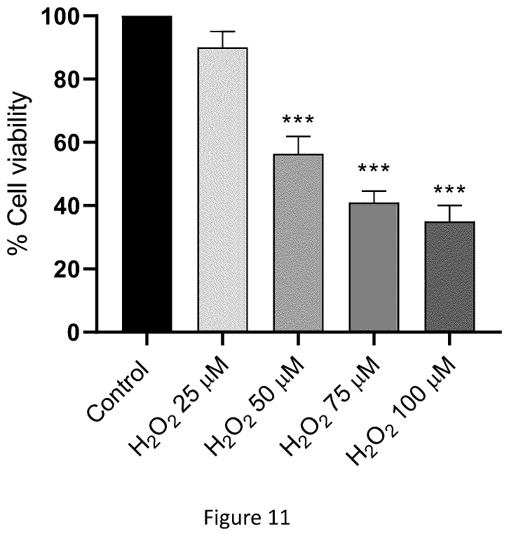

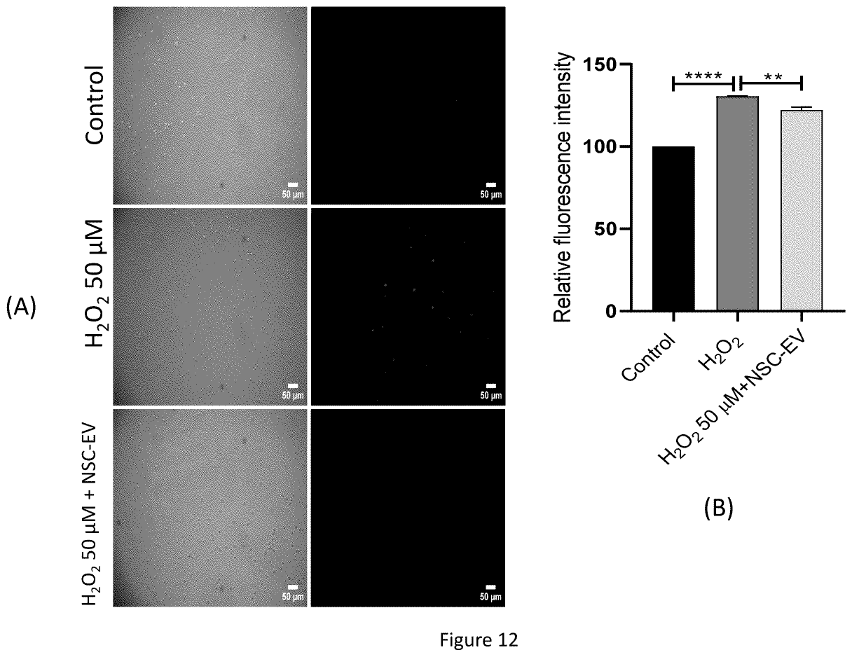

Oxidative stress occurs when the levels of reactive oxygen species (ROS) exceed the antioxidant capacity of stem cells, resulting in senescence and cell death. Hydrogen peroxide (H2O2) is one of the ROS produced during cell metabolism and has been shown to activate numerous mechanisms that decrease survival and induce apoptosis in various cell types, including NSCs. To identified the concentration and duration of treatment with H2O2 capable of causing moderate damage (50% cell death), cells were seeded under differentiation conditions and the medium was supplemented with different concentrations of H2O2. After 30 minutes, the medium containing H2O2 was replaced, and the cells were incubated for 24 hours with a fresh medium. Finally, the MTT assay was performed according to Mosmann, T., 1983. Based on the results obtained (FIG. 11), it was selected a concentration of H2O2 of 50 μM for subsequent experiments. To confirm that treatment with H2O2 (50 μM) generates oxidative stress, experiments were conducted to evidence the presence of ROS using 2′,7′-dichlorofluorescein diacetate (DCFH DA) probe according to Gomes, A, 2005. As observed in FIG. 12, under damage conditions, green fluorescence is detected due to ROS generation, indicating that H2O2 (50 μM) generates oxidative stress.

### Example 8: Effect of NSC-EVs on NSC Proliferation Under Oxidative Stress Conditions

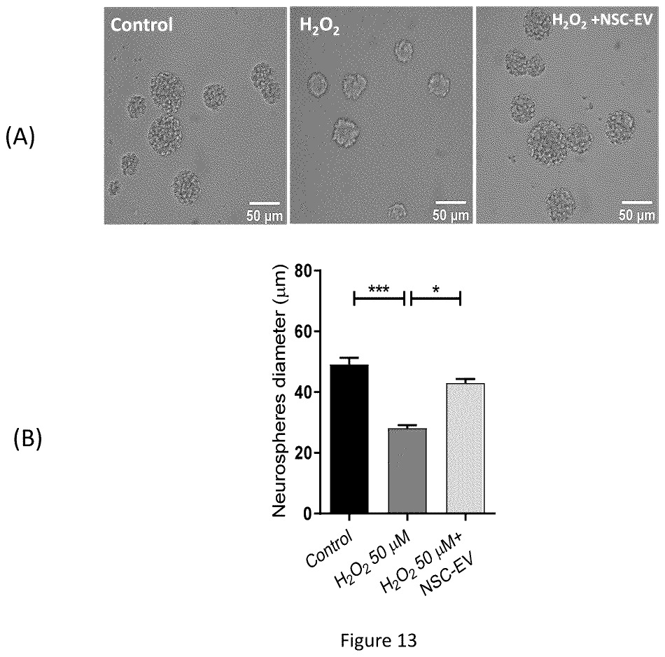

The proliferation of NSCs under oxidative damage conditions (treatment with 50 μM H2O2) was assessed in the presence and absence of NSC EV from the present invention. For this, cells incubated under proliferation conditions were treated for 30 minutes with 50 μM H2O2. After this period, a fresh medium supplemented with NSC-EVs from the present invention or PBS (control) was added, and the cells were kept under proliferation conditions for 96 hours. Photos were taken of 20 randomly chosen fields, and the diameter of the neurospheres was quantified. As seen in the photographs and the resulting graph from the quantification (FIG. 13), under oxidative damage conditions, there is a decrease in the diameter of the neurospheres, suggesting that the ability of NSCs to proliferate is affected. Interestingly, cells treated with NSC-EVs, even in the presence of H2O2, show a clear recovery in the diameter of the neurospheres, reaching values similar to the control condition (FIG. 13).

### Example 9: Effect of NSC-EV Treatment on NSCs Differentiation Under Oxidative Stress Conditions

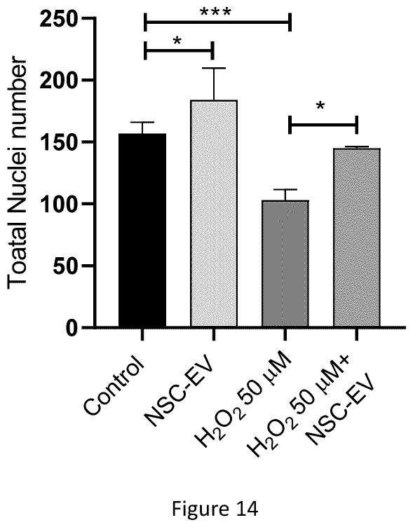

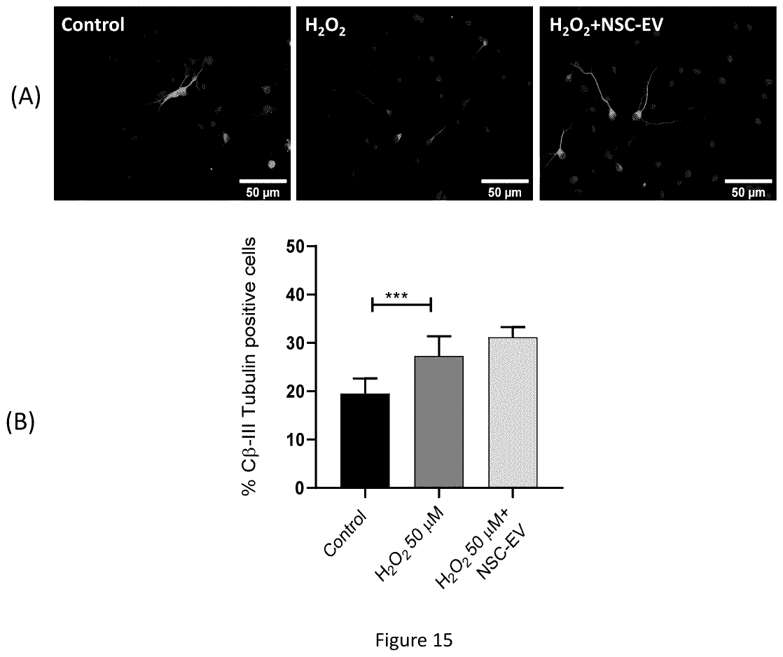

The differentiation of NSCs into functional neurons in stressful environments is essential for repairing damaged tissue. To assess the neuronal differentiation capacity of NSCs under oxidative stress conditions and the effect of NSC-EVs, NSCs were sown under differentiation conditions as previously mentioned and incubated with 50 μM H2O2 for 30 minutes. After this period, the medium was renewed, and fresh medium containing NSC-EV or PBS (control) was added. After 72 hours, an immunocytochemistry followed by fluorescence microscopy analysis was performed, and the percentage of β-III Tubulin positive cells (neurons) relative to the total number of nuclei stained with Hoechst was quantified. As shown in FIG. 14, measured as a significant decrease in the total number of nuclei compared to the control condition, confirming the detrimental effect of oxidative stress on NSCs. Interestingly, under this damage condition, supplementing the culture medium with NSC-EVs restores this parameter to values comparable to the control, showing that the extracellular vesicles of the present invention increase cell survival even under damage conditions. The resulting neuronal differentiation analysis from the quantification of the percentage of β-III Tubulin positive cells indicates that H2O2 treatment induces an increase in NSC differentiation towards the neuronal lineage (FIG. 15). However, under this stress condition, treatment with the indicated concentration of the NSC-EVs, according to the present invention, does not affect this process.

### Example 10: Effect of NSC-EVs from the Present Invention on Morphological and Functional Parameters Associated with Neuronal Maturation and Synapses Under Oxidative Stress Conditions

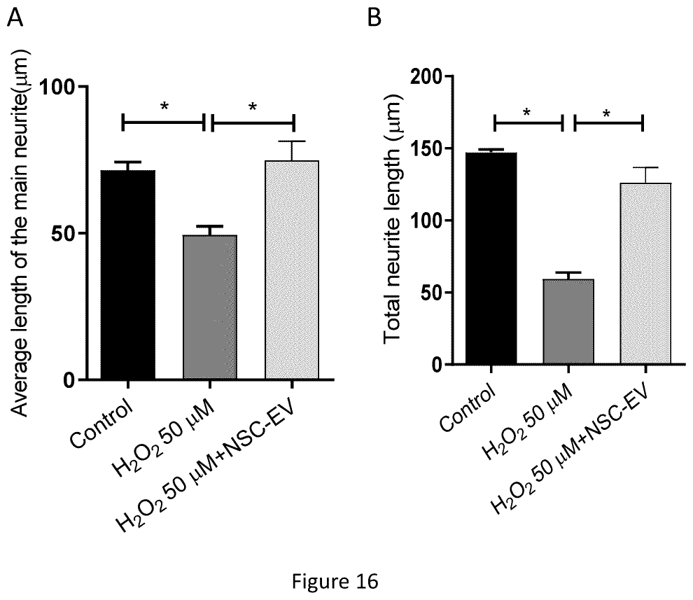

A detailed morphological study was conducted in which it was observed that under oxidative stress conditions, neurons develop shorter neurites; with a decrease in the average length of the main neurite, as well as a shorter total neurite length compared to the control condition (FIG. 16). Under this condition, treatment of the culture with the composition comprising extracellular vesicles of the present invention restores neuronal morphology, recovering neurite length to values similar to the neuronal control, as seen in FIG. 16. Based on these results, which show that supplementing the culture medium with NSC-EV from the present invention restores the proliferation capacity of NSCs and morphology affected by oxidative stress, and considering that treatment with 50 μM H2O2 induces the accumulation of ROS; the inventors decided to evaluate the effect of NSC-EV on this parameter. Similarly, as previously described, NSCs were sown under differentiation conditions and incubated for 30 minutes with H2O2 (50 μM). Subsequently, the medium was removed, and 7.5 μg of NSC-EV, obtained according to Example 2, or PBS as a negative control was added, and the cells were incubated for 1 hour. After this period, the DCFH DA probe was added to the culture for an extra 30 minutes. Finally, a fluorescence microscopic analysis was performed. As seen in FIG. 12, supplementation with 7.5 μg of NSC-EV from the present invention reduces fluorescence compared to the condition of treatment with 50 μM H2O2, suggesting that such extracellular vesicles derived from stem cells reduce the generation or accumulation of ROS in target cells.

### Example 11: Establishment of the In Vitro Inflammatory Stress Model

Neuroinflammation occurs in response to various degenerative disorders or CNS injuries. Microglia, the resident macrophages of the brain, and macrophages migrating from the periphery, play a significant role in the development of inflammation. When activated, they release pro-inflammatory cytokines such as nitric oxide, TNFα, and IL-1, which affect the survival and differentiation of NSCs. To study the effect of the inflammatory environment on stem cell proliferation and differentiation, the inventors used an in vitro inflammation model previously established in the lab in which NSCs are treated with 20% (V/V) medium from Raw 293 macrophages previously activated with 1 μg/ml of LPS. This creates a condition of moderate to severe damage in the NSCs.

### Example 12: Effect of NSC-EVs on the Proliferation and Differentiation of NSCs Under Inflammatory Stress Conditions

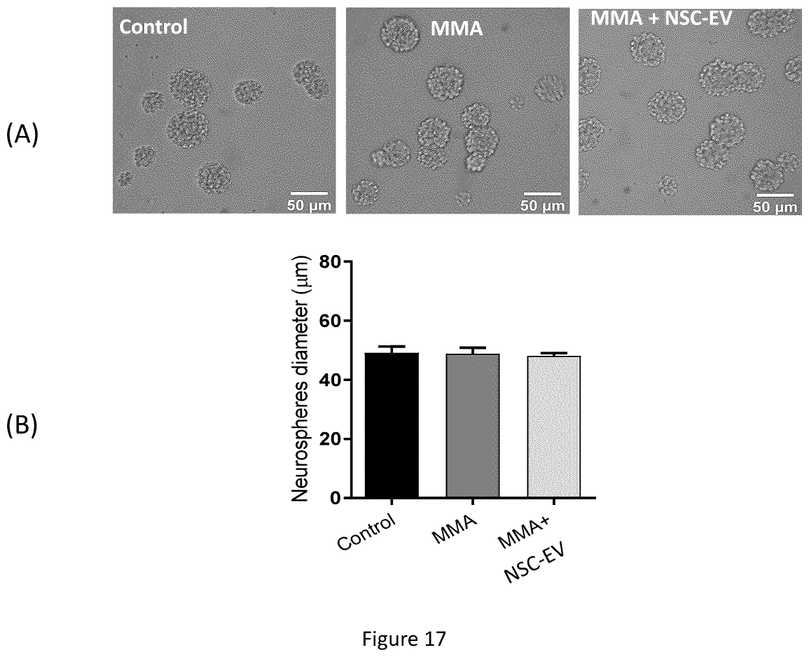

To assess the effect of inflammatory stress on the proliferation of NSCs and the impact of treatment with NSC-EV of the present invention under this condition, cells were incubated under proliferation conditions with 20% (V/V) of AMM as previously described, in the presence or absence of NSC-EV of the present invention (7.5 μg of proteins) for 96 hours. Photos were taken from 20 randomly chosen fields, and the diameter of the neurospheres was quantified. As seen in FIG. 17, treatment with AMM does not modify the diameter of the neurospheres, and under this condition, the treatment with the indicated concentration of the NSC-EV of the present invention does not affect this parameter. Therefore, under inflammatory stress conditions, NSC-EV of the present invention does not alter the proliferation of neural stem cells.

A common characteristic of various CNS conditions that cause acute or chronic inflammation is neuronal death. Considering our results showing that treatment with NSC-EV of the present invention improves neuronal differentiation and induces favorable morphological and functional changes under control conditions, the inventors assessed whether the NSC-EVs, which are part of the composition of the present invention, could have a beneficial effect on neuronal differentiation under inflammatory stress.

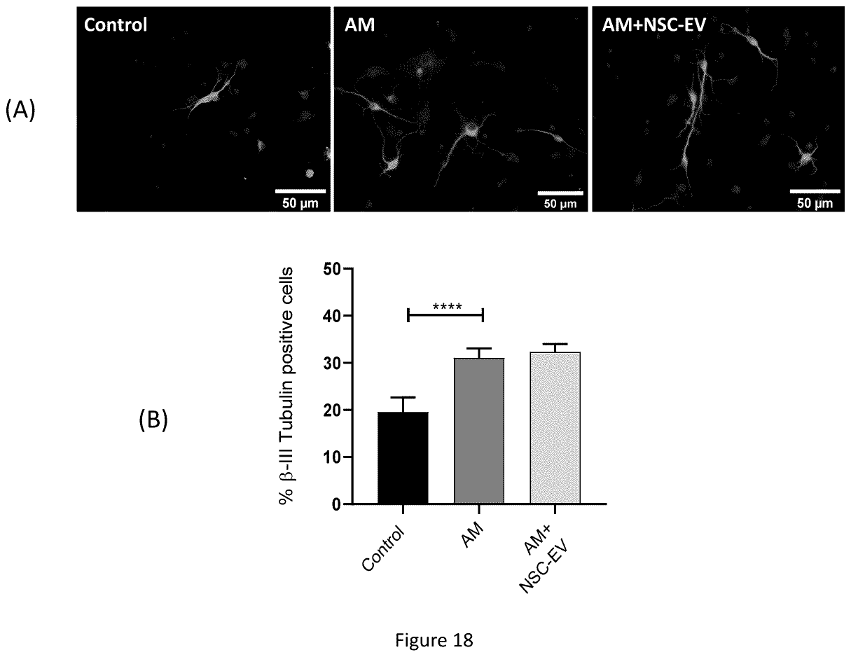

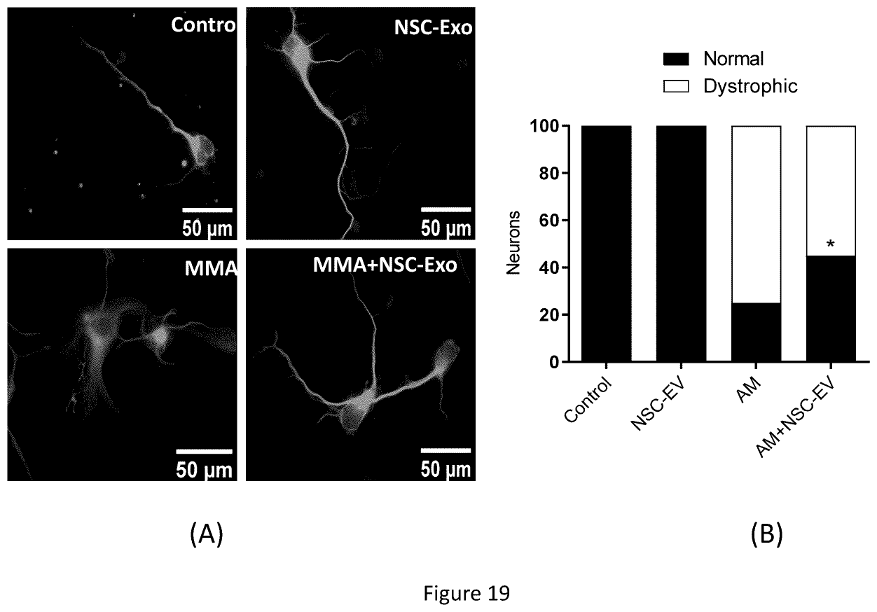

To evaluate neuronal differentiation capacity under inflammatory stress conditions and study the effect of NSC EVs in this damage situation, NSCs were seeded under differentiation conditions and incubated for 72 hours with 20% V/V of AMM in the presence or absence of NSC-EV of the present invention (7.5 μg of proteins). Subsequently, immunocytochemistry was performed, and the percentage of β-III Tubulin-positive cells was quantified. The analysis shows that under inflammatory damage conditions, neuronal differentiation is induced (FIG. 18). However, upon a more detailed examination of neuronal morphology, it can be observed that the resulting neurons are aberrant, showing an apparent disorganization of tubulin, dystrophy, increased soma size, and the presence of cytoplasmic vacuoles (FIG. 19).

### Example 13: Effect of NSC-EVs on Neuronal Morphology and Functionality Under Damage Conditions

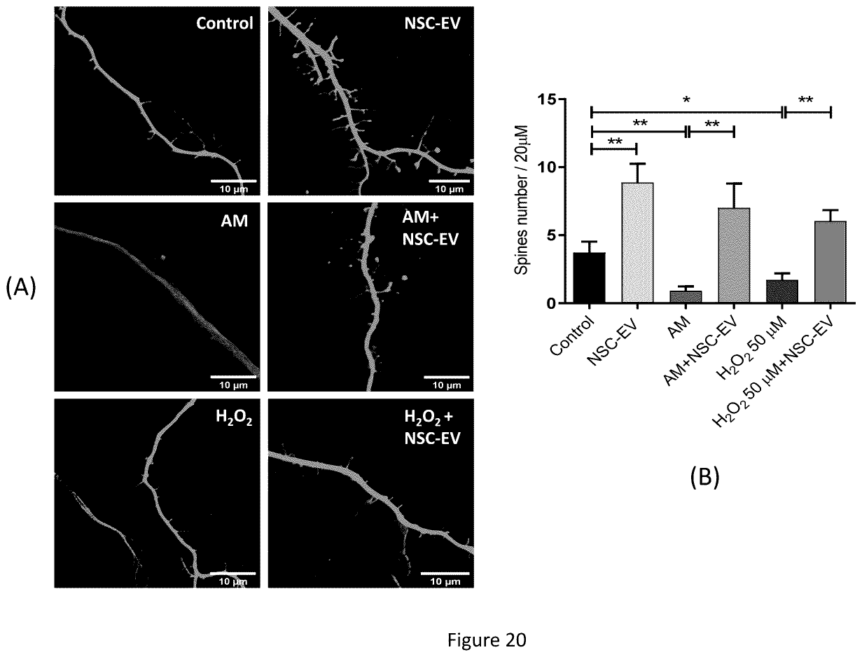

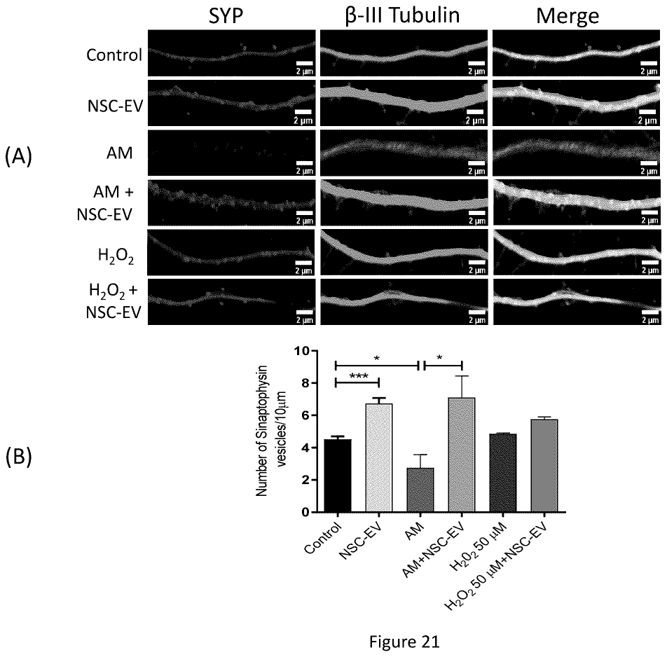

Both tissue damage conditions (oxidative stress and inflammatory processes) affect the differentiation of NSCs into functional neurons. Interestingly, treatment with NSC-EV from the current invention reverses the morphology of neurons affected by both inflammation and oxidative stress. To further characterize these effects and considering that morphology is linked to neuronal function, parameters such as dendritic spine density, the number of vesicles containing SYP, and expression levels of proteins SYP and PSD 95 were evaluated. The same protocol described previously was followed, which consists of incubating 250,000 cells under differentiation conditions in the presence of 50 μM of H2O2 or 20% (V/V) of AMM to model oxidative or inflammatory stress, respectively, with or without 7.5 μg of NSC EV proteins. After 72 hours, immunocytochemistry was performed with anti β-III Tubulin and anti SYP antibodies following the described protocols. For analysis, 10 photos from randomly selected fields were taken using the confocal microscope (Zeiss LSM 880) and analyzed with the ImageJ software. The number of dendritic spines in 20 μm of neurite length and the number of vesicles containing SYP in 10 μm of neurite length were quantified. As shown in FIG. 20, both stress conditions significantly reduce dendritic spine density. However, when analyzing this parameter in cells incubated in the presence of NSC EV, a recovery is observed, reaching values comparable to the control (FIG. 20). Quantifying the number of vesicles containing SYP in 10 μm of neurite length indicates that inflammatory damage negatively affects this parameter, measured as a significant decrease in the number of vesicles positive for SYP (FIG. 21). Interestingly, when cells incubated under this damage condition are treated with NSC-EV from the current invention, this parameter significantly recovers, reaching values similar to the control condition (FIG. 21). These results allow us to conclude that supplementing the culture medium with NSC-EV from the current invention under stress conditions reverses or prevents the deleterious effect of the damage measured in morphological and functional parameters related to neuronal maturation and synaptic functionality.

### Example 14: Proteomic Study of NSC-EV Included in the Composition of the Present Invention

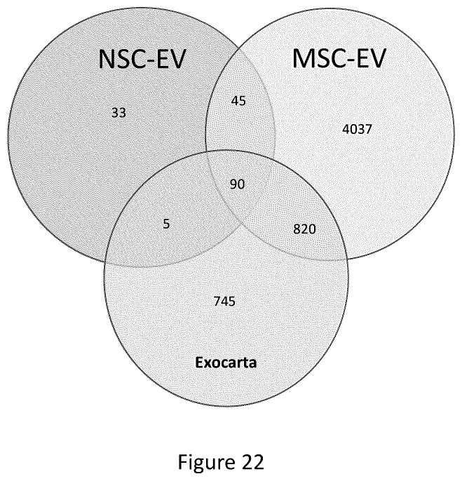

Given that proteins are fundamental components for the inducer effect of neuronal differentiation promoted by NSC-EVs, the inventors conducted a proteomic study using tandem mass spectrometry (LC-MS/MS). To do this, the extracellular vesicles of the current invention were purified, obtained according to example 2 (Three biological replicas), and they were processed using the MaxQuant Perseus software (Max Planck Institute). 173 proteins were identified with an error limit of less than 1% (FDR<1%, from the English term False Discovery Rate). With the aim of identifying specific proteins present in the NSC-EV of the current invention that are not present in EVs derived from other cell types, a comparative analysis was performed. To do this, the inventors compared the proteins identified in the NSC-EV of the current invention with the following protein databases:
1. All mouse proteins from the Exocarta database (http://exocarta.org/). Exocarta is a specialized database that compiles detailed information about proteins identified in exosomes from different cell types and physiological or pathological conditions. From this list, those corresponding to NSCs were excluded.
2. The proteins found in exosomes derived from mesenchymal stem cells (MSCs). These were downloaded from the PRIDE repository project PXD027041 (https://www.ebi.ac.uk/pride/archive/project s/PXD027041) and the data used correspond to the proteins of exosomes from MSCs in the control group.
3. The results shown in FIG. 22 demonstrate that, of the 173 proteins identified in the NSC-EV of the current invention, 33 proteins are specific to EVs derived from this cell type.

From the total proteins identified, a manual search was carried out in the literature, and using the functional annotations of the Uniprot database, the inventors grouped the proteins into the following categories:
1. Extracellular vesicle marker proteins
2. Proteins involved in the proliferation, differentiation of NSCs, and neuronal maturation
3. Proteins in response to oxidative and inflammatory stress

As can be seen in Table 1, 9 proteins were identified that are involved in the biogenesis and secretion of exosomes. Some of these proteins, such as TSG 101, CD9, CD81, and HSP 90, are considered exosome markers.

TABLE 1
Proteins involved in the biogenesis and secretion of exosomes
identified through mass spectrometry analysis in NSC-EV.

| Protein | Gene | Uniprot ID |
|---|---|---|
| Heat shock protein HSP 90-alpha | Hsp90aa1 | P07901 |
| Heat shock protein HSP 90-beta | Hsp90ab1 | P11499 |
| CD81 antigen | Cd81 | P35762 |
| CD9 antigen | Cd9 | P40240 |
| Tumor susceptibility gene 101 protein | Tsg101 | Q61187 |
| Multivesicular body subunit 12B | MVB12B | Q9H7P6 |
| Vacuolar protein sorting-associated protein 37B | VPS37B | Q9H9H4 |
| Charged multivesicular body protein 4b | Chmp4b | Q9D8B3 |
| Programmed cell death 6-interacting protein | Pdcd6ip | Q9WU78 |
| (also known as ALIX) | | |

Additionally, the NSC-EVs contain numerous proteins involved in the proliferation and differentiation of NSCs, as well as neuronal maturation, growth, and synaptic function (Table 2)

TABLE 2
Proteins involved in the proliferation and differentiation
of NSCs, as well as neuronal maturation and synapse, identified
through mass spectrometry analysis in NSC-EV.

| Protein | Gene | Uniprot ID |
|---|---|---|
| Neural cell adhesion molecule 1 | Ncam 1 | P13595 |
| Neuronal cell adhesion molecule | Nrcam | Q810U4 |
| Neuronal membrane glycoprotein M6-b | Gpm6b | P35803 |
| Dihydropyrimidinase-related protein 3 | DPYSL3 | Q14195 |
| Laminin subunit beta-1 | Lamb1 | P02469 |
| Dihydropyrimidinase-related protein 2 | Dpysl2 | O08553 |
| Syntenin-1 | Sdcbp | O08992 |
| Fibroblast growth factor | Fgf2 | Q925A1 |
| 2′,3′-cyclic-nucleotide 3′- | Cnp | P16330 |
| phosphodiesterase | | |
| AP-2 complex subunit alpha-1 | Ap2a1 | P17426 |
| MARCKS-related protein | Marcksl1 | P28667 |
| Neuronal membrane glycoprotein M6-a | Gpm6a | P35802 |
| Neurogenic differentiation factor 6 | Neurod6 | P48986 |
| 14-3-3 protein epsilon | Ywhae | P62259 |
| Pleiotrophin | Ptn | P63089 |
| 14-3-3 protein zeta/delta | Ywhaz | P63101 |
| Epidermal growth factor receptor | Egfr | Q01279 |
| Glia-derived nexin | Serpine2 | Q07235 |
| Delphilin | Grid2ip | Q0QWG9 |
| Brevican core protein | Bcan | Q61361 |
| Chondroitin sulfate proteoglycan 5 | Cspg5 | Q71M36 |
| Phosphatidylinositol-binding clathrin | Picalm | O55012 |
| assembly protein | | |
| Tenascin | Tnc | Q80YX1 |
| Tubulin beta-2B chain | Tubb2b | Q9CWF2 |
| Neurocan core protein | Ncan | P55066 |

It was demonstrated that the NSC-EV of the present invention exert a protective and reparative effect on neuronal morphology and function even under oxidative and inflammatory stress conditions. In this regard, as observed in Table 3, the NSC-EVs of the present invention also contain proteins involved in the cellular response to oxidative stress and inflammation.

TABLE 3
Proteins involved in defense against oxidative and inflammatory stress
identified through mass spectrometry analysis in the NSC-EV.

| Protein | Gene | Uniprot ID |
|---|---|---|
| Vascular cell adhesion molecule 1 | Vcam 1 | Q3UPN1 |
| Glutathione S-transferase kappa 1 | Gstk1 | A0A0N4SVE5 |
| Annexin A1 | Anxa1 | P10107 |
| Thioredoxin | Txn | P10639 |
| Catalase | Cat | P24270 |
| Reticulon-3 | Rtn3 | Q9ES97 |
| Peroxiredoxin-2 | Prdx2 | Q61171 |

### Example 15: Establishment of Stable Cell Lines Overexpressing α-Synuclein Variants

For the generation of stable cell lines overexpressing both wild-type and A53T variants of the α-Synuclein protein, SH-SY5Y cells were transfected with the pcDNA3-WT-α-Synuclein or pcDNA3-A53T-α-Synuclein plasmids, along with the pSuper-GFP plasmid NEOMYCIN RESISTAN. Subsequently, stable clones were selected with neomycin (G418).

Following this trial, and validated by western blot assays and immunocytochemistry coupled with fluorescence microscopy analysis, stable clones SH-SY5Y pcDNA3-WT-α-Synuclein (WT1 and WT2) and pcDNA3-A53T-α-Synuclein (A53T1 and A53T2) were selected and further investigated. These clones displayed higher levels of α-Synuclein protein compared to control cells transfected with the pcDNA3 vector (pcDNA3 clon-1 and pcDNA3 clon-2).

Parkinson's disease is characterized by the overexpression of Synuclein that forms Lewy bodies, an increase in ROS species and finally dopaminergic neuronal death. To validate the established model, the inventors measured cell viability by MTT (FIG. 23A), characterized the type of cell death (FIG. 23 B) and measured the amount of ROS species (FIG. 23 C). The inventors demonstrated that Synuclein overexpression induces human neuroblastoma cell death by increasing apoptosis and ROS accumulation.

### Example 16: Human Neuroblastoma Cells Line Take Mouse NSC-EVs

To evaluate that human cell line, incorporate mouse NSC-EV, the inventors labelled them with DilC probe. For labelling, 25 ul of NSC-EVs in PBS were mixed with Vybrant™ DiI Cell-Labelling Solution (Thermo Fisher) (1:9) and incubated at room temperature for 30 min. Next, Dil-NSC-EVs were added to human neuroblastoma cells and the uptake of NSC-EVs was immediately monitored by in vivo confocal microscopy (Zeiss LSM 880) and qualitative analyses were performed with Image J” (NIH. The inventors confirmed the uptake of mouse-NSC-EV by human neuroblastoma cells (FIG. 2).

### Example 17: Effect of Mouse NSC-EV on the In Vitro Parkinson's Disease Model Based on α-Synuclein Overexpression in Human Neurobalstome Cell SHsY5Y

To assess whether the NSC-EVs of the current invention impact the cellular viability of cells overexpressing α-synuclein, the clones, derived as per Example 15, were grown under differentiation conditions in the presence or absence of 2,5, 7,5. 10, 20, 40 and 50 μg of NSC-EV for 72 hours. Following this period, an MTT assay was performed. The results showed that the NSC-EVs of this invention significantly increase cellular viability, specifically with 5-10 μg of NSC-EVs) both in control cells and in clones overexpressing α-synuclein (FIG. 23A y 29). These findings suggest that NSC-EVs enhance cell viability, but more importantly, they have a neuroprotective effect, increasing the survival of cells overexpressing α-synuclein.

Additionally, NSC-EVs from this invention significantly reduce ROS in both control cells and clones overexpressing α-synuclein (FIG. 22 B). These findings suggest that NSC-EVs have a neuroprotective effect on the survival of cells overexpressing α-synuclein, which might be directly related to the reduction in ROS accumulation.

### Example 18: Establishment of the In Vitro Parkinson's Disease Model with 60H-Dopamine in the Human Neuroblastoma SH-SY5Y Cell Line

The 6-OH-dopamine (6-OHD) is a neurotoxin similar to the neurotransmitter dopamine, providing selectivity for dopaminergic neurons. Inside cells, it oxidizes, producing ROS, causing mitochondrial dysfunction, and activating pro-apoptotic molecular factors that lead to cell death. These characteristics make it a suitable compound for generating neuronal damage models that mimic Parkinson's Disease.

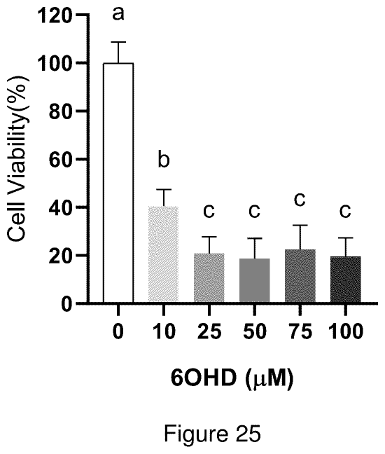

To determine the drug concentration that induces moderate culture damage, an MTT assay was conducted on cultures for 72 hours with increasing concentrations of 6-OHD. As seen in FIG. 25, all tested concentrations of 60HD decrease cell viability. Moreover, treating the culture with 10 μM of 60HD causes moderate cell death, making this concentration representative of moderate disease damage (FIG. 25).

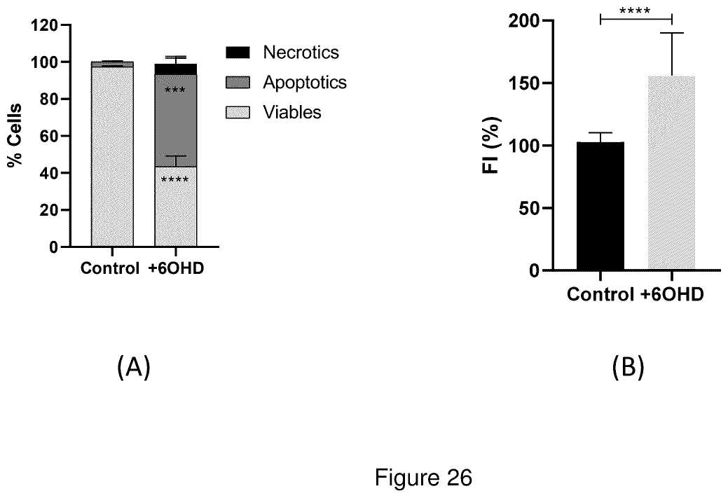

To characterize the type of cell death the drug induces, BE/NA double-staining assays were performed on cells treated with 10 μM of 60HD. As shown in FIG. 26 A, treatment with 60HD results in an increase in apoptotic cells, indicating the drug reduces cell viability by inducing apoptosis or programmed cell death. Regarding necrosis, there's a non-significant increase in necrotic cell percentage.

Continuing the analysis, oxidative stress was quantified using the DCFH-DA probe in the presence of 10 μM of 6OHD. As shown in FIG. 26 B, a significant increase in ROS was detected in cells treated with the drug compared to control. Thus, it can be concluded that 60HD increases the accumulation of intracellular ROS.

### Example 19: Effect of NSC-EV Neuroprotection in an In Vitro Parkinson's Disease Model Induced by 60H-Dopamine in the Human Neuroblastoma SH-SY5Y Cell Line

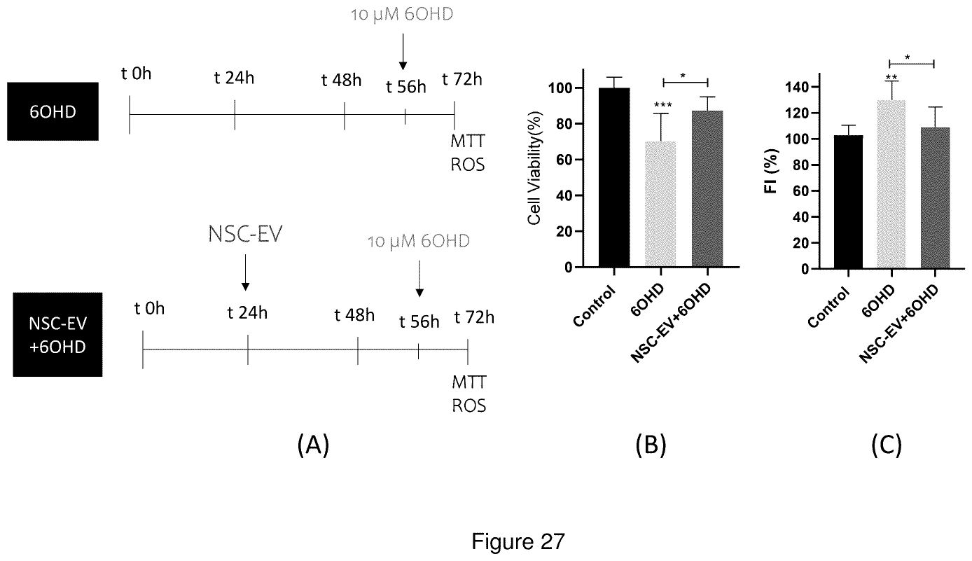

To evaluate whether the NSC-EVs of this invention could protect and restore cells against oxidative damage caused by 6OHDA, two experimental strategies were employed. In the first, the impact of cell incubation with NSC-EVs prior to damage on viability was assessed (FIG. 27 A). After the incubation period, an MTT assay was conducted, and ROS was quantified using the DCFH-DA probe. The results showed that pre-incubating the cells with NSC-EVs significantly increases cell viability compared to cells not pre-incubated with control NSC-EV (FIG. 27B). These findings suggest that NSC-EVs have a neuroprotective effect, enhancing the survival of cells treated with 60HD. This potential protective effect was further investigated by pre-incubating cells with NSC-EVs, followed by 60HD treatment and ROS quantification using the DCFH-DA probe. The results showed that pre-incubating the cells with NSC-EVs significantly reduces ROS compared to the control (FIG. 27C), suggesting that NSC-EVs provide neuroprotection against oxidative damage.

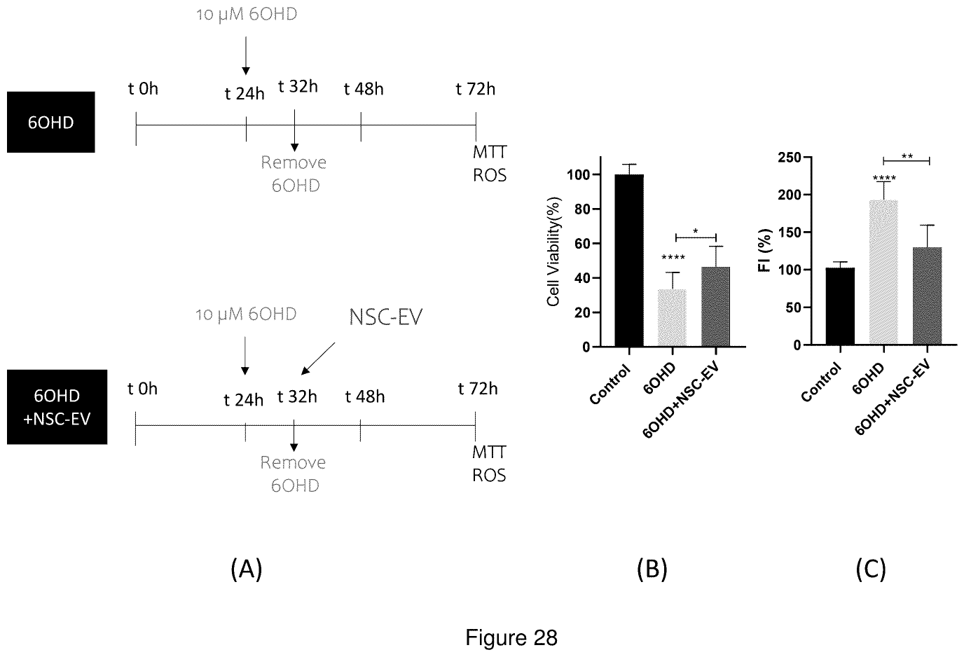

In the second strategy, the effect of NSC-EV treatment after damage on viability was evaluated (FIG. 28A). After the incubation period, an MTT assay was performed, and ROS was quantified using the DCFH-DA probe. The results showed that cells incubated with NSC-EVs post oxidative damage displayed significantly increased cell viability compared to cells not treated with EVs (FIG. 28B). These findings suggest that NSC-EVs have a neurorestorative effect, enhancing the survival of cells previously treated with 60HD. Cells treated with NSC-EVs post-damage induction exhibited lower ROS amounts (FIG. 28C), suggesting that NSC-EVs have a neurorestorative effect following oxidative damage.

### REFERENCES

1—Aktas, O., Ullrich, O., Infante-Duarte, C., Nitsch, R., and Zipp, F. (2007). Neuronal damage in brain inflammation. Archives of neurology 64(2), 185-189.
2—Andreotti, J. P., Silva, W. N., Costa, A. C., Picoli, C. C., Bitencourt, F. C., Coimbra-Campos, L. M., et al. (Year). “Neural stem cell niche heterogeneity”, in: Seminars in cell & developmental biology: Elsevier), 42-53.
3—Aranda, A., Sequedo, L., Tolosa, L., Quintas, G., Burello, E., Castell, J., et al. (2013). Dichloro-dihydro-fluorescein diacetate (DCFH-DA) assay: a quantitative method for oxidative stress assessment of nanoparticle-treated cells. Toxicology in vitro 27(2), 954-963.
4—Cossetti, C., Alfaro-Cervello, C., DonegA, M., Tyzack, G., and Pluchino, S. (2012). New perspectives of tissue remodelling with neural stem and progenitor cell-based therapies. Cell and tissue research 349(1), 321-329.
5—Costa, M. R., Wen, G., Lepier, A., Schroeder, T., and Götz, M. (2008). Par-complex proteins promote proliferative progenitor divisions in the developing mouse cerebral cortex. Development 135.
6—Desai, A. R., and McConnell, S. K. (2000). Progressive restriction in fate potential by neural progenitors during cerebral cortical development. Development 127(13), 2863-2872.
7—Eggenhofer, E., Benseler, V., Kroemer, A., Popp, F., Geissler, E., Schlitt, H., et al. (2012). Mesenchymal stem cells are short-lived and do not migrate beyond the lungs after intravenous infusion. Frontiers in immunology 3, 297.
8—Fan W J, Yan M C, Wang L, Sun Y Z, Deng J B, Deng J X. Synaptic aging disrupts synaptic morphology and function in cerebellar Purkinje cells. Neural Regen Res. 2018 June; 13(6):1019-1025.
9—Fischer, R., and Maier, O. (2015). Interrelation of oxidative stress and inflammation in neurodegenerative disease: role of TNF. Oxidative medicine and cellular longevity 2015.
10—Gomes, A., E. Fernandes, and J. L. Lima (2005), Fluorescence probes used for detection of reactive oxygen species. Journal of biochemical and biophysical methods, 65 (2 3): p. 45-80.
11—Han, C., Sun, X., Liu, L., Jiang, H., Shen, Y., Xu, X., et al. (2016). Exosomes and their therapeutic potentials of stem cells. Stem cells international 2016.
12—Harman, D. (2006). Alzheimer's disease pathogenesis: role of aging. Annals of the New York Academy of Sciences 1067(1), 454-460.
13—Johnstone, R. M. (2006). Exosomes biological significance: a concise review. Blood Cells, Molecules, and Diseases 36(2), 315-321.
14—Lai, R. C., Arslan, F., Lee, M. M., Sze, N. S. K., Choo, A., Chen, T. S., et al. (2010). Exosome secreted by MSC reduces myocardial ischemia/reperfusion injury. Stem cell research 4(3), 214-222.
15—Lin, H. J., Wang, X., Shaffer, K. M., Sasaki, C. Y., and Ma, W. (2004). Characterization of H2O2-induced acute apoptosis in cultured neural stem/progenitor cells. Febs Letters 570(1-3), 102-106.
16—Mathieu, M., Martin-Jaular, L., Lavieu, G., and Théry, C. (2019). Specificities of secretion and uptake of exosomes and other extracellular vesicles for cell-to-cell communication. Nature cell biology 21(1), 9-17.
17—Montaner, A., da Silva Santana, T. T., Schroeder, T., Einicker-Lamas, M., Girardini, J., Costa, M. R., et al. (2018). Specific phospholipids regulate the acquisition of neuronal and astroglial identities in post-mitotic cells. Scientific reports 8(1), 1-13.
18—Mosmann, T. (1983). Rapid colorimetric assay for cellular growth and survival: application to proliferation and cytotoxicity assays. Journal of immunological methods 65(1-2), 55-63.
19—Mijalkov, M., Volpe, G., Fernaud-Espinosa, I. et al. Dendritic spines are lost in clusters in Alzheimer's disease. Sci Rep 11, 12350 (2021).
20—Ottoboni, L., von Wunster, B., and Martino, G. (2020). Therapeutic Plasticity of Neural Stem Cells. Frontiers in Neurology 11. doi: 10.3389/fneur.2020.00148.
21—Reik, W. (2007). Stability and flexibility of epigenetic gene regulation in mammalian development. Nature 447(7143), 425-432.
22—Rietze, R. L., and Reynolds, B. A. (2006). Neural stem cell isolation and characterization. Methods in enzymology 419, 3-23.
23—Saxena, S., Mathur, A., and Kakkar, P. (2019). Critical role of mitochondrial dysfunction and impaired mitophagy in diabetic nephropathy. Journal of cellular physiology 234 (11), 19223-19236.
24—Trounson, A., and McDonald, C. (2015). Stem cell therapies in clinical trials: progress and challenges. Cell stem cell 17(1), 11-22.
25—Uttara, B., Singh, A. V., Zamboni, P., and Mahajan, R. (2009). Oxidative stress and neurodegenerative diseases: a review of upstream and downstream antioxidant therapeutic options. Current neuropharmacology 7(1), 65-74.
26—Watanabe, T., and Raff, M. C. (1990). Rod photoreceptor development in vitro: intrinsic properties of proliferating neuroepithelial cells change as development proceeds in the rat retina. Neuron 4(3), 461-467.
27—Witwer, K. W., Buzis, E. I., Bemis, L. T., Bora, A., Lässer, C., Lötvall, J., et al. (2013). Standardization of sample collection, isolation and analysis methods in extracellular vesicle research. Journal of extracellular vesicles 2(1), 20360.
28—Xiao, L., Saiki, C., and Ide, R. (2014). Stem cell therapy for central nerve system injuries: glial cells hold the key. Neural regeneration research 9(13), 1253.
29—Xin, H., Li, Y., Buller, B., Katakowski, M., Zhang, Y., Wang, X., et al. (2012). Exosome-mediated transfer of miR-133b from multipotent mesenchymal stromal cells to neural cells contributes to neurite outgrowth. Stem cells 30(7), 1556-1564.
30—Yokoi, A., and Ochiya, T. (Year). “Exosomes and extracellular vesicles: rethinking the essential values in cancer biology”, in: Seminars in Cancer Biology: Elsevier), 79-91.
31—Zhang, Q., Li, J., An, W., Fan, Y., and Cao, Q. (2020). Neural stem cell secretome and its role in the treatment of neurodegenerative disorders. J. Integr. Neurosci 19(1), 179-185.

1. A composition for the treatment of neurological diseases comprising extracellular vesicles derived from stem cell selected from the group consisting of animal stem cell and cell line.

2. The composition of claim 1, wherein said animal stem cells is selected from the group consisting of neural stem cells, mesenchymal stem cells, embryonic stem cells, and umbilical stem cells.

3. The composition of claim 1, wherein said animal stem cells are neural stem cells.

4. The composition of claim 3, wherein said neural stem cells are selected from the group consisting of stem cells from brain regions consisting of the lateral portion of the dorsal telencephalon (cortex) of fetal brain tissue, the subventricular zone (SVZ), and the hippocampus.

5. The composition of claim 1, wherein said cell lines are selected from the group consisting of neural stem cell lines.

6. The composition of claim 1, wherein said extracellular vesicles are derived from neural stem cells.

7. The composition of claim 6, wherein said neural stem cells are derived from mouse neural stem cells.

8. The composition of claim 1, wherein said extracellular vesicles come from human stem cell line.

9. The composition of claim 1, wherein said extracellular vesicles derived from stem cells are neural stem cells and have a size between 50 nm and 400 nm.

10. (canceled)

11. (canceled)

12. The composition of claim 1, wherein said extracellular vesicles comprise proteins selected from the group consisting of TSG-101, HSP70, ALIX, HSP90, CD81, and CD9.

13. The composition of claim 12, wherein said extracellular vesicles further comprise at least one protein selected from the group consisting of Multivesicular body subunit 12B, Vacuolar protein sorting-associated protein 37B, and Charged multivesicular body protein 4b.

14. (canceled)

15. The composition of claim 12, wherein said extracellular vesicles further comprise at least one protein selected from the group consisting of Neural cell adhesion molecule 1, Neuronal cell adhesion molecule, Neuronal membrane glycoprotein M6-b, Dihydropyrimidinase-related protein 3, Laminin subunit beta-1, Dihydropyrimidinase-related protein 2, Syntenin-1, Fibroblast growth factor, 2′,3′-cyclic-nucleotide 3′-phosphodiesterase, AP-2 complex subunit alpha-1, MARCKS-related protein, Neuronal membrane glycoprotein M6-a, Neurogenic differentiation factor 6, 14-3-3 protein épsilon, Pleiotrophin, 14-3-3 protein zeta/delta, Epidermal growth factor receptor, Glia-derived nexin, Delphilin, Brevican core protein, Chondroitin sulfate proteoglycan 5, Phosphatidylinositol binding clathrin assembly protein, Tenascin, Tubulin beta-2B chain, and Neurocan core protein.

16. The composition of claim 12, wherein said extracellular vesicles further comprise the proteins selected from the group consisting of: Neural cell adhesion molecule 1, Neuronal cell adhesion molecule, Neuronal membrane glycoprotein M6-b, Dihydropyrimidinase-related protein 3, Laminin subunit beta-1, Dihydropyrimidinase-related protein 2, Syntenin-1, Fibroblast growth factor, 2′,3′-cyclic-nucleotide 3′-phosphodiesterase, AP-2 complex subunit alpha-1, MARCKS-related protein, Neuronal membrane glycoprotein M6-a, Neurogenic differentiation factor 6, 14-3-3 protein épsilon, Pleiotrophin, 14-3-3 protein zeta/delta, Epidermal growth factor receptor, Glia-derived nexin, Delphilin, Brevican core protein, Chondroitin sulfate proteoglycan 5, Phosphatidylinositol binding clathrin assembly protein, Tenascin, Tubulin beta-2B chain, and Neurocan core.

17. The composition of claim 12, wherein said extracellular vesicles further comprise at least one protein selected from the group consisting of Vascular cell adhesion molecule 1, Glutathione S-transferase kappa 1, Annexin A1, Thioredoxin, Catalase, Reticulon-3, and Peroxiredoxin-2.

18. The composition of claim 12, wherein said extracellular vesicles further comprise the proteins selected from the group consisting of: Vascular cell adhesion molecule 1, Glutathione S-transferase kappa 1, Annexin A1, Thioredoxin, Catalase, Reticulon-3, and Peroxiredoxin-2.

19. The composition of claim 1, wherein said extracellular vesicles comprise the proteins selected form the group consisting of TSG-101, HSP70, ALIX, HSP90, CD81, CD9, Multivesicular body subunit 12B, Vacuolar protein sorting-associated protein 37B, Charged multivesicular body protein 4b, Neural cell adhesion molecule 1, Neuronal cell adhesion molecule, Neuronal membrane glycoprotein M6-b, Dihydropyrimidinase-related protein 3, Laminin subunit beta-1, Dihydropyrimidinase-related protein 2, Syntenin-1, Fibroblast growth factor, 2′,3′-cyclic-nucleotide 3′-phosphodiesterase, AP-2 complex subunit alpha-1, MARCKS-related protein, Neuronal membrane glycoprotein M6-a, Neurogenic differentiation factor 6, 14-3-3 protein épsilon, Pleiotrophin, 14-3-3 protein zeta/delta, Epidermal growth factor receptor, Glia-derived nexin, Delphilin, Brevican core protein, Chondroitin sulfate proteoglycan 5, Phosphatidylinositol binding clathrin assembly protein, Tenascin, Tubulin beta-2B chain, Neurocan core protein, Vascular cell adhesion molecule 1, Glutathione S-transferase kappa 1, Annexin A1, Thioredoxin, Catalase, Reticulon-3, and Peroxiredoxin-2.

20. An extracellular vesicle derived from neural stem cells wherein said extracellular vesicle comprises the proteins selected from the group consisting of TSG-101, HSP70, ALIX, HSP90, CD81, CD9, Multivesicular body subunit 12B, Vacuolar protein sorting-associated protein 37B, Charged multivesicular body protein 4b, Neural cell adhesion molecule 1, Neuronal cell adhesion molecule, Neuronal membrane glycoprotein M6-b, Dihydropyrimidinase-related protein 3, Laminin subunit beta-1, Dihydropyrimidinase-related protein 2, Syntenin-1, Fibroblast growth factor, 2′,3′-cyclic-nucleotide 3′-phosphodiesterase, AP-2 complex subunit alpha-1, MARCKS-related protein, Neuronal membrane glycoprotein M6-a, Neurogenic differentiation factor 6, 14-3-3 protein epsilon, Pleiotrophin, 14-3-3 protein zeta/delta, Epidermal growth factor receptor, Glia-derived nexin, Delphilin, Brevican core protein, Chondroitin sulfate proteoglycan 5, Phosphatidylinositol binding clathrin assembly protein, Tenascin, Tubulin beta-2B chain, Neurocan core protein, Vascular cell adhesion molecule 1, Glutathione S-transferase kappa 1, Annexin A1, Thioredoxin, Catalase, Reticulon-3, and Peroxiredoxin-2.

21. The extracellular vesicle according to claim 20, wherein its size is between 50 nm and 400 nm.

22. (canceled)

23. A composition that promotes the proliferation of neural stem cells, neuronal differentiation, and/or neurite development and/or dendritic development comprising the extracellular vesicles according to claim 20.

24. A pharmaceutical formulation that promotes the proliferation of neural stem cells, neuronal differentiation, and/or neurite development and/or dendritic development, wherein said pharmaceutical formulation comprises the composition of claim 1.

25. A method for treating neurological diseases comprising administering the composition of claim 1, wherein said diseases are selected from the group consisting of Amyotrophic Lateral Sclerosis, Parkinson's, Alzheimer's, dementia, Huntington disease, Lafora disease, Schizophrenia, Autism, stroke, and from the peripheral nervous system: diabetes, post-chemotherapy paresthesia and pathologies associated with motor, sensory and autonomic nerves.

26. A method for treating neurological diseases, wherein said method comprises administering the extracellular vesicles according to claim 22, wherein said diseases are selected from the group consisting of Amyotrophic Lateral Sclerosis, Parkinson's, Alzheimer's, dementia, Huntington disease, Lafora disease, Schizophrenia, Autism, and from the peripheral nervous system: diabetes, post-chemotherapy paresthesia and pathologies associated with motor, sensory and autonomic nerves.

27. A method for treating neurodegenerative diseases comprising administering to a mammalian the composition of claim 1, wherein said disease is selected from the group consisting of Parkinson's and strokes.

28. (canceled)

29. (canceled)

30. A method to obtain extracellular vesicles (EV) according to claim 20, comprising culturing the said neural stem cells or cell lines, in a proliferating medium, collecting supernatant, and collecting the fractions containing said extracellular vesicles.

31. The method, according to claim 30, wherein said stem cell is a neural stem cell from brain tissue selected from the group consisting of a lateral portion of the dorsal telencephalon (cortex) of fetal brain tissue, subventricular zone (SVZ), and the hippocampus tissue.

32. The method, according to claim 31, wherein said method comprises the steps of:
a) isolating a lateral portion of the brain tissue, b) chemically and mechanically disrupting brain tissue into single cells, c) centrifuging, removing the supernatant and resuspending the pellet in serum-free medium, d) culturing cells in a proliferating medium in a culture flask, e) after 7 days, replating cells to obtain a second passage, f) collecting supernatant and centrifuging and filtering to remove cell debris, g) concentrating, separating, and purifying by size exclusion chromatography and h) collecting and pooling fractions and characterizing said neural stem cell-derived extracellular vesicles.

33. The method according to claim 31, wherein said neural stem cell is a mouse neural stem cell.

34. (canceled)

35. The method according to claim 33, wherein said cell line is selected from the group consisting of ReNcell and Human Neural stem cell line.
